SEQUENCE LISTING (42 sequences)

SEQ ID NO: 1 - protein, 378 aa, Mus musculus
MPYSNLHPAIPRPRGHRSKYVALIFLVASLMILWVAKDPPNHTLKYLALHLASHELGLLLKNLCCLAEELCHVQSRYQGSYWKAVRACLGCPIHCMAMILLSSYFYFLQNTADIYLSWMFGLLVLYKSLSMLLGLQSLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 2 - protein, 379 aa, Homo sapiens [sequence omitted]

SEQ ID NO: 3 - protein, 241 aa, Mus musculus
LTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 4 - protein, 241 aa, Homo sapiens
LAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 5 - protein, 232 aa, Mus musculus
LTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 6 - protein, 232 aa, Homo sapiens
LAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 7 - protein, 122 aa, synthetic construct
QVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGG

SEQ ID NO: 8 - protein, 132 aa, synthetic construct
QVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGG

SEQ ID NO: 9 - protein, 123 aa, synthetic construct
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGG

SEQ ID NO: 10 - protein, 127 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGG

SEQ ID NO: 11 - protein, 394 aa, synthetic construct
  UNSURE 1..31: note = optionally absent
MGHHHHHHDYDIPTTENLYFQGSDYKDDDDKQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 12 - protein, 363 aa, synthetic construct
QVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 13 - protein, 404 aa, synthetic construct
  UNSURE 1..31: note = optionally absent
MGHHHHHHDYDIPTTENLYFQGSDYKDDDDKQVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 14 - protein, 373 aa, synthetic construct
QVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 15 - protein, 395 aa, synthetic construct
  UNSURE 1..31: note = optionally absent
MGHHHHHHDYDIPTTENLYFQGSDYKDDDDKMAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 16 - protein, 364 aa, synthetic construct
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 17 - protein, 399 aa, synthetic construct
  UNSURE 1..31: note = optionally absent
MGHHHHHHDYDIPTTENLYFQGSDYKDDDDKQVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 18 - protein, 368 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 19 - protein, 385 aa, synthetic construct
  UNSURE 1..31: note = optionally absent
MGHHHHHHDYDIPTTENLYFQGSDYKDDDDKQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 20 - protein, 354 aa, synthetic construct
QVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 21 - protein, 395 aa, synthetic construct
  UNSURE 1..31: note = optionally absent
MGHHHHHHDYDIPTTENLYFQGSDYKDDDDKQVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 22 - protein, 364 aa, synthetic construct
QVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 23 - protein, 386 aa, synthetic construct
  UNSURE 1..31: note = optionally absent
MGHHHHHHDYDIPTTENLYFQGSDYKDDDDKMAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 24 - protein, 355 aa, synthetic construct
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 25 - protein, 390 aa, synthetic construct
  UNSURE 1..31: note = optionally absent
MGHHHHHHDYDIPTTENLYFQGSDYKDDDDKQVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 26 - protein, 359 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQ

SEQ ID NO: 27 - protein, 394 aa, synthetic construct
  UNSURE 1..31: note = optionally absent
MGHHHHHHDYDIPTTENLYFQGSDYKDDDDKQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 28 - protein, 363 aa, synthetic construct
QVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 29 - protein, 404 aa, synthetic construct
  UNSURE 1..31: note = optionally absent
MGHHHHHHDYDIPTTENLYFQGSDYKDDDDKQVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 30 - protein, 373 aa, synthetic construct
QVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 31 - protein, 395 aa, synthetic construct
  UNSURE 1..31: note = optionally absent
MGHHHHHHDYDIPTTENLYFQGSDYKDDDDKMAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 32 - protein, 364 aa, synthetic construct
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 33 - protein, 398 aa, synthetic construct
  UNSURE 1..31: note = optionally absent
MGHHHHHHDYDIPTTENLYFQGSDYKDDDDKQVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDF

SEQ ID NO: 34 - protein, 367 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDF

SEQ ID NO: 35 - protein, 385 aa, synthetic construct
  UNSURE 1..31: note = optionally absent
MGHHHHHHDYDIPTTENLYFQGSDYKDDDDKQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 36 - protein, 354 aa, synthetic construct
QVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 37 - protein, 395 aa, synthetic construct
  UNSURE 1..31: note = optionally absent
MGHHHHHHDYDIPTTENLYFQGSDYKDDDDKQVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 38 - protein, 364 aa, synthetic construct
QVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 39 - protein, 386 aa, synthetic construct
  UNSURE 1..31: note = optionally absent
MGHHHHHHDYDIPTTENLYFQGSDYKDDDDKMAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 40 - protein, 355 aa, synthetic construct
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 41 - protein, 390 aa, synthetic construct
  UNSURE 1..31: note = optionally absent
MGHHHHHHDYDIPTTENLYFQGSDYKDDDDKQVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 42 - protein, 359 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

(19) United States
(12) Patent Application Publication
(10) Pub. No.: US 2023/0331795 A1
(43) Pub. Date: Oct. 19, 2023

(54) ENGINEERING IMMUNE CHECKPOINT BLOCKADE NANOBODY-STING DELTA-TM FUSION PROTEIN AS A VERSATILE TREATMENT FOR SOLID TUMOR CANCERS

(71) Applicant: Massachusetts Institute of Technology, Cambridge, MA (US)

(72) Inventors: Angela M. BELCHER, Cambridge, MA (US); Paula T. HAMMOND, Cambridge, MA (US); Yanpu HE, Cambridge, MA (US); Celestine HONG, Cambridge, MA (US); Shengnan HUANG, Cambridge, MA (US); Jiahe LI, Cambridge, MA (US)

(21) Appl. No.: 18/312,030

(22) Filed: May 4, 2023

Related U.S. Application Data
(63) Continuation of application No. 17/181,884, filed on Feb. 22, 2021.
(60) Provisional application No. 62/979,733, filed on Feb. 21, 2020.

(51) Int. Cl. C07K 14/47; C07K 16/28; A61K 45/06; A61K 31/7084

(52) U.S. Cl. CPC C07K 14/4702; C07K 16/2818; C07K 16/2827; A61K 45/06; A61K 31/7084; A61K 38/00

(57) ABSTRACT

Disclosed are fusion proteins including a STINGΔTM protein fused to a nanobody, and compositions thereof that further include a STING agonist. Also disclosed are methods of treating cancer, which is achieved by administering said compositions.

### RELATED APPLICATIONS

This application is a continuation in part of U.S. Application Ser. No. 17/181,884 filed Feb. 22, 2021, which claims the benefit of priority to U.S. Provisional Patent Application Ser. No. 62/979,733, filed Feb. 21, 2020, each of which is incorporated by reference herein in its entirety

### SEQUENCE LISTING

A computer readable form of the Sequence Listing is filed with this application by electronic submission and is incorporated into this application by reference in its entirety. The Sequence Listing is contained in the file created on Apr. 18, 2023 having the file name “23-0366-US-CIP.xml” and is 57,441 bytes in size.

### BACKGROUND

Activation of the stimulator of interferon genes (STING) pathway through cyclic dinucleotides (CDNs) could be used as a potent vaccine adjuvant against infectious diseases as well as to increase tumor immunogenicity towards cancer immunotherapy in solid tumors. Despite the promise of CDNs, such as cGAMP, as immune adjuvants, they suffer from several limitations: (1) CDNs exhibit fast clearance from the injection site, which may induce systemic toxicity; (2) naturally derived CDNs are susceptible to enzymatic degradation, which can lower the efficacy of adjuvanticity potential; and (3) CDNs have inefficient intracellular transport properties due to limited endosomal escape or reliance on the expression of a specific transporter protein. Hence, there is an urgent need to find new strategies for delivering CDNs.

### SUMMARY

In one aspect, the present disclosure provides a composition comprising a fusion protein comprising a STINGΔTM protein fused to a nanobody. Numerous embodiments are further provided that can be applied to any aspect of the present invention described herein. For example, in some embodiments, the STINGΔTM comprises an amino acid sequence with at least 75%, 80%, 85%, 90%, 95%, 96%, 97%, 98%, 99% or 100% homology to the amino acid sequence selected from SEQ ID NOs: 3-6. In some embodiments, the nanobody is fused to the N-terminus of the STINGΔTM. In some embodiments, the nanobody is capable of binding to a cancer cell or or tumor extracellular matrices. In some embodiments, the nanobody is capable of binding to Cytotoxic T-Lymphocyte Associated Protein 4 (CTLA4), Programmed death protein 1 (PD-1), Programmed death-ligand 1 (PD-L1), Programmed death-ligand 2 (PD-L2), adenosine A2a receptor (A2AR), B7 homolog 3 protein (B7-H3), B7 homolog 4 protein (B7-H4), B- and T-lymphocyte attenuator (BTLA), killer cell immunoglobulin-like receptor (KIR), Lymphocyte-activation gene 3 (LAG3), T cell immunoglobulin and mucin domain-containing protein 3 (TIM-3), Fibronectin extra type III B module (EIIIB), T cell immunoreceptor with Ig and ITIM domains (TIGIT), Natural killer group 2, member A (NGK2A), P-selectin glycoprotein ligand-1 (PSGL-1), or V-domain immunoglobulin suppressor of T-cell activation (VISTA). In one embodiment, the nanobody comprises an amino acid sequence with at least 75%, 80%, 85%, 90%, 95%, 96%, 97%, 98%, 99% or 100% identity to the amino acid sequence selected from the group consisting of SEQ ID NO: 7-10. In another embodiment, the fusion protein comprises an amino acid sequence with at least 75%, 80%, 85%, 90%, 95%, 96%, 97%, 98%, 99% or 100% identity to the amino acid sequence selected from SEQ ID NO: 11-42, wherein residues in parentheses are optional and may be present or may be deleted in whole or in part.

In further embodiments, the disclosure provides nucleic acids encoding the fusion protein of any embodiment or combination of embodiments herein, expression vectors comprising the nucleic acid operatively linked to a suitable control sequence, and host cells comprising the fusion protein, nucleic acid, and/or expression vector of any embodiment or combination of embodiments disclosed herein.

In another aspect, the disclosure provides compositions, comprising the fusion protein of any embodiment or combination of embodiments herein and a STING agonist. In one embodiment, the STING agonist is a cytosolic cyclic dinucleotide (CDN). In another embodiment, the CDN is c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP. In a further embodiment, the STING agonist is a non-nucleotidyl small molecule. In various embodiments, the non-nucleotidyl small molecule is 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo[3,2-a:3′,2′-d]pyrazine-5,10(5aH,10aH)-dione, 4-(2-chloro-6-fluorobenzyl)-N-(furan-2-yl methyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3-b][1,4]thiazine-7-carboxamide, 2-oxo-1,2,3,4-tetrahydroquinoline-7-carboxamide, or 2-Oxo-1,2,3,4-tetrahydroquinazoline-7-carboxamides.

In another aspect, the disclosure provides methods of treating cancer or an infectious disease, comprising administering to a patient in need thereof an effective amount of the composition of any embodiment or combination of embodiments herein. In one embodiment, the method is for treating cancer, incuding but not limited to a colon carcinoma, a melanoma, or breast cancer.

### BRIEF DESCRIPTION OF THE DRAWINGS

FIG. 1A-1F. ICB-STINGΔTM-cGAMP in vitro function and in vivo biodistribution. (FIG. 1A) Protein homology-modeling structural prediction of dimerized anti-PDL1-STINGΔTM protein complexed with a cGAMP molecule with SWISS modeling. (FIG. 1B) HEK293T interferon-luciferase reporter cells (N=3) treated with different ICB-ΔTM-cGAMP complexes, along with S365AΔTM mutant controls. Luciferase activity was determined 24 hours post treatment. (FIG. 1C) Ex vivo assay of OT1 CD8+ T cells' cytotoxicity against SIINFEKL-MC38 cancer cells, ICB-ΔTM-cGAMP complexes along with equal molar amount of anti-PDL1 antibody were added during the co-culture of CD8+ T cells and cancer cells, the cytotoxicity was measured with flow cytometry with representative plot shown in (FIG. 1D). (FIG. 1E) Representative IVIS images of biodistribution (FIG. 1F) of Cy5-labeled ICB-ΔTM-cGAMP complexes 24 hours post intratumoral injections (tdLN for tumor draining lymph node and cLN for contralateral lymph node). Data were analyzed with one-way ANOVA.

FIG. 2A-2I. ICB-STINGΔTM-cGAMP effectively eliminates subcutaneous MC38 tumors. (FIG. 2A) Groups of C57BL/6 mice (N=10) were inoculated with 1 million MC38 cells subcutaneously on the hind right flank and then treated intratumorally with 4 doses of ICB-ΔTM-cGAMP complexes along with GFP-nanobody and no cGAMP controls. (FIG. 2B) Tumor growth and (FIG. 2C) survival of mice were monitored, all surviving mice (blocks meaning the mouse has been euthanized after its tumor burden exceeded 1000 mm³) were tumor free as shown in (FIG. 2E) and successfully rejected a secondary rechallenge of 0.1 million MC38 cells subcutaneously injected on the hind left flank (FIG. 2D). (FIG. 2F) A new experiment was performed to analyze the tumor microenvironment, groups of C57BL/6 mice (N=5) were inoculated with 1 million MC38 cells subcutaneously then treated intratumorally with 3 doses of ICB-ΔTM-cGAMP complexes along with anti-GFP-ΔTM-cGAMP, no cGAMP, and commercial anti-PDL1&CTLA4 antibody controls. (FIG. 2G) Percentage body weight change of mice since the start of treatment, same figure legend as (I). (FIG. 2H) Cytokine/chemokine levels and (FIG. 2I) immune cell compositions in the tumors were quantified on Day 17. Tumor volume and body weight change were analyzed with two-way ANOVA, the survival curve was analyzed with Kaplan-Meier, the remaining plots were analyzed with one-way ANOVA.

FIG. 3A-3J. ICB-STINGΔTM-cGAMP effectively eliminates subcutaneous YUMM1.7 tumors. (FIG. 3A) Groups of C57BL/6 mice (N=5 for no cGAMP and αGFP-ΔTM-cGAMP groups, N=8 for αPDL1&CTLA4 mAb group, N=10 for the remaining three groups) were inoculated with 1 million YUMM1.7 cells subcutaneously on the hind right flank and then treated intratumorally with 5 doses of ICB-ΔTM-cGAMP complexes along with controls. (FIG. 3B) Tumor growth and (FIG. 3C) survival of mice were monitored, all surviving mice (blocks meaning the mouse has been euthanized after its tumor burden exceeded 1000 mm3) were tumor free (FIG. 3E) and successfully rejected a secondary rechallenge of 0.1 million YUMM1.7 cells subcutaneously injected on the hind left flank except for one mouse from the αPDL1&CTLA4 mAb group (FIG. 3D). (FIG. 3F) A new experiment was performed to analyze the tumor microenvironment, groups of C57BL/6 mice (N=5) were inoculated with 1 million YUMM1.7 cells subcutaneously then treated with 3 doses of ICB-ΔTM-cGAMP complexes along with controls. (FIG. 3G) Percentage body weight change of mice since the start of treatment, figure legend same as (FIG. 3I, FIG. 3J). (FIG. 3H) Cytokine/chemokine levels, (FIG. 3I) immune cell compositions (FIG. 3J) intracellular cytokine staining of NK and NKT cells in the tumors were quantified on Day 15. Tumor volume and body weight change were analyzed with two-way ANOVA, the survival curve was analyzed with Kaplan-Meier, the remaining plots were analyzed with one-way ANOVA.

FIG. 4A-4L. Cellular pathways governing ICB-STINGΔTM-cGAMP therapeutic efficacy. (FIG. 4A) Groups of C57BL/6 mice (N=10) were each depleted of macrophages, CD4⁺ T cells, CD8⁺ T cells, NK cells with intraperitoneal antibody injections, along with two groups with no depletion (untreated N=10, treated N=8), then inoculated with YUMM1.7 tumors and treated intratumorally with 2 doses of ΔPDL1&CTLA4-ΔTM-cGAMP. (FIG. 4B) Tumor growth and (FIG. 4C) survival of mice were monitored. (FIG. 4D) On day 23 post tumor inoculation, an IFNy ELISPOT assay was performed with mice PBMCs against X-ray irradiated YUMM1.7 cells, with representative wells of each group showing in (FIG. 4E). SFU represents spot forming unit. (FIG. 4F) Cy5-labelled ICB-ΔTM protein complexed with cGAMP were injected into YUMM1.7 tumors on C57BL/6 mice. 24 hours post injection, tumors were harvested, digested and analyzed with flow cytometry to show colocalization of cell types with ICB-ΔTM-cGAMP. (FIG. 4G) Ex vivo C57BL/6 splenocyte stimulation with ΔPDL1&CTLA4-ΔTM-cGAMP complex followed by intracellular cytokine staining for IFNγ in CD4⁺ T cells. (FIG. 4H) Ex vivo isolated CD4⁺ T cell stimulation with ΔPDL1&CTLA4-ΔTM-cGAMP complex followed by intracellular cytokine staining for IFNγ. (FIG. 4I) Confocal micrographs of isolated CD4⁺ T cells incubated with Cy5-labelled ΔPDL1&CTLA4-ΔTM-cGAMP, top and bottom images are representative cells interacting with ICB-ΔTM-cGAMP from the same treatment group. Scale bar=10 μm. (FIG. 4J) Proposed cellular mechanism of CD4⁺ T cells as first responders that interact with ICB-ΔTM-cGAMP complexes, followed by activation of NK-mediated cytotoxicity and priming of CD8⁺ T cells with dendritic cells. Tumor antigens released from NK- and CD8-mediated cytotoxicity are captured and presented by dendritic cells to sustain the immunity cycle. Tumor volume and body weight change were analyzed with two-way ANOVA, the survival curve was analyzed with Kaplan-Meier; the remaining plots were analyzed with one-way ANOVA.

FIG. 5A-5G. ICB-STINGΔTM-cGAMP controls metastasis in 4T1 model. (FIG. 5A) Groups of Balb/c mice (N=10) were inoculated with 1 million 4T1 cells in the mammary fat pad and then treated intratumorally with 3 doses of ICB-ΔTM-cGAMP complexes along with controls, on Day 15 the tumors were surgically removed. (FIG. 5B) Tumor growth and (FIG. 5C) survival of mice were monitored, the surviving mice was tumor free. (FIG. 5D) Percentage body weight change of mice since the start of treatment. (FIG. 5E) Cytokine/chemokine levels, (FIG. 5F) immune cell compositions (FIG. 5G) CD4⁺ T cell phenotyping from excised tumors on Day 15. Tumor volume and body weight change were analyzed with two-way ANOVA, the survival curve was analyzed with Kaplan-Meier; the remaining plots were analyzed with one-way ANOVA.

FIG. 6A-6C. ICB-STINGΔTM-cGAMP demonstrates the potential to overcome host STING deficiency. (FIG. 6A) Groups of STING KO mice (N=8) were inoculated with 1 million MC38 cells subcutaneously in the hind right flank and then treated intratumorally with 4 doses of αPDL1&CTLA4-ΔTM-cGAMP complexes along with controls. (FIG. 6B) shows the tumor growth curve. On Day 12 the tumors were removed for cytokine/chemokine analysis (FIG. 6C). Tumor volume change were analyzed with two-way ANOVA.

FIG. 7A-7D. (FIG. 7A) Pilot study (N=14) treating MC38 tumors comparing ΔPDL1-STINGΔTM+cGAMP with the STINGΔTM+cGAMP system. (FIG. 7B) Tumor growth and (FIG. 7C) survival of mice were monitored, along with (FIG. 7D) photos of all mice at Day 16, 20, 34, and 70 (blocks meaning the mouse has been euthanized after its tumor burden exceeded 1000 mm³).

FIG. 8. Mice MC38 tumor photos of FIG. 2A treatment at Day 25 (blocks meaning the mouse has been euthanized after its tumor burden exceeded 1000 mm³).

FIG. 9. Mice YUMM1.7 tumor photos of FIG. 3A treatment at Days 16 and 37 (blocks meaning the mouse has been euthanized after its tumor burden exceeded 1000 mm³).

FIG. 10. Mice YUMM1.7 tumor photos of FIG. 4A depletion study at Days 11, 16, 20 and 65 (blocks meaning the mouse has been euthanized after its tumor burden exceeded 1000 mm³).

FIG. 11A-11C. Groups of C57BL/6 mice (N=5) bearing YUMM1.7 tumors were sacrificed 24 hours after the first intratumoral treatment. Tumors were digested to analyze the (FIG. 11A) immune cell composition, (FIG. 11B) NK, NKT and (FIG. 11C) CD4⁺ T cell phenotypes.

### DETAILED DESCRIPTION

In one aspect, the present disclosure provides fusion proteins comprising STINGΔTM protein fused to a nanobody. The fusion proteins of the disclosure can be used, for example in methods to treat cancer or infectious disease, as disclosed herein. The cytosolic domain of STING protein (STINGΔTM) is fused to a nanobody, which can for example be complexed with a STING agonist for use in the therapeutic methods disclosed herein.

The term “STING”, also known as stimulator of interferon genes (STING). STING is a protein that in humans is encoded by the STING1 gene. STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection by binding to the same cell that secretes it (autocrine signaling) and nearby cells (paracrine signaling.)

Below are non-limiting examples of STING proteins.

Mouse STING
(SEQ ID NO: 1)
MPYSNLHPAI PRPRGHRSKY VALIFLVASL MILWVAKDPP
NHTLKYLALH LASHELGLLL KNLCCLAEEL CHVQSRYQGS
YWKAVRACLG CPIHCMAMIL LSSYFYFLQN TADIYLSWMF
GLLVLYKSLS MLLGLQSLTP AEVSAVCEEK KLNVAHGLAW
SYYIGYLRLI LPGLQARIRM FNQLHNNMLS GAGSRRLYIL
FPLDCGVPDN LSVVDPNIRF RDMLPQQNID RAGIKNRVYS
NSVYEILENG QPAGVCILEY ATPLQTLFAM SQDAKAGFSR
EDRLEQAKLF CRTLEEILED VPESRNNCRL IVYQEPTDGN
SFSLSQEVLR HIRQEEKEEV TMNAPMTSVA PPPSVLSQEP
RLLISGMDQP LPLRTDLI
Human STING
(SEQ ID NO: 2)
MPHSSLHPSI PCPRGHGAQK AALVLLSACL VTLWGLGEPP
EHTLRYLVLH LASLQLGLLL NGVCSLAEEL RHIHSRYRGS
YWRTVRACLG CPLRRGALLL LSTYFYYSLP NAVGPPFTWM
LALLGLSQAL NILLGLKGLA PAEISAVCEK GNFNVAHGLA
WSYYIGYLRL ILPELQARIR TYNQHYNNLL RGAVSQRLYI
LLPLDCGVPD NLSMADPNIR FLDKLPQQTG DRAGIKDRVY
SNSIYELLEN GORAGTCVLE YATPLQTLFA MSQYSQAGFS
REDRLEQAKL FCRTLEDILA DAPESQNNCR LIAYQEPADD
SSFSLSQEVL RHLRQEEKEE VTVGSLKTSA VPSTSTMSQE
PELLISGMEK PLPLRTDFS
Mouse STINGΔTM
(SEQ ID NO: 3)
LTP AEVSAVCEEK KLNVAHGLAW SYYIGYLRLI LPGLQARIRM
FNQLHNNMLS GAGSRRLYIL FPLDCGVPDN LSVVDPNIRF
RDMLPQQNID RAGIKNRVYS NSVYEILENG QPAGVCILEY
ATPLQTLFAM SQDAKAGFSR EDRLEQAKLF CRTLEEILED
VPESRNNCRL IVYQEPTDGN SFSLSQEVLR HIRQEEKEEV
TMNAPMTSVA PPPSVLSQEP RLLISGMDQ LPLRTDLI
Human STINGΔTM
(SEQ ID NO: 4)
LA PAEISAVCEK GNENVAHGLA WSYYIGYLRL ILPELQARIR
TYNQHYNNLL RGAVSQRLYI LLPLDCGVPD NLSMADPNIR
FLDKLPQQTG DRAGIKDRVY SNSIYELLEN GQRAGTCVLE
YATPLQTLFA MSQYSQAGFS REDRLEQAKL FCRTLEDILA
DAPESQNNCR LIAYQEPADD SSFSLSQEVL RHLRQEEKEE
VTVGSLKTSA VPSTSTMSQE PELLISGMEK PLPLRTDFS
Mouse STINGΔTMΔC9
(SEQ ID NO: 5)
LTP AEVSAVCEEK KLNVAHGLAW SYYIGYLRLI LPGLQARIRM
FNQLHNNMLS GAGSRRLYIL FPLDCGVPDN LSVVDPNIRF
RDMLPQQNID RAGIKNRVYS_NSVYEILENG QPAGVCILEY
ATPLQTLFAM SQDAKAGFSR EDRLEQAKLF CRTLEEILED_
VPESRNNCRL IVYQEPTDGN SFSLSQEVLR HIRQEEKEEV
TMNAPMTSVA PPPSVLSQEP_RLLISGMDQP
Human STINGΔTMΔC9
(SEQ ID NO: 6)
LA PAEISAVCEK GNENVAHGLA WSYYIGYLRL ILPELQARIR
TYNQHYNNLL RGAVSQRLYI LLPLDCGVPD NLSMADPNIR
FLDKLPQQTG DRAGIKDRVY SNSIYELLEN GQRAGTCVLE
YATPLQTLFA MSQYSQAGFS REDRLEQAKL FCRTLEDILA
DAPESQNNCR LIAYQEPADD SSFSLSQEVL RHLRQEEKEE
VTVGSLKTSA VPSTSTMSQE PELLISGMEK

In one embodiment, the STINGΔTM protein comprises an amino acid sequence with at least 75%, 80%, 85%, 90%, 95%, 96%, 97%, 98%, 99% or 100% identity to the amino acid sequence selected from SEQ ID NO: 3-6.

The STINGΔTM protein and nanobody may be directly linked in the fusion protein, or may be separated by an amino acid linker. Any suitable amino acid linker may be used. The nanobody may be fusion N-terminal to the STINGΔTM, or may be fused C-terminal to the STINGΔTM.

Any nanobody as suitable for an intended use may be present in the fusion proteins. A nanobody is a single-domain antibody (sdAb); an antibody fragment consisting of a single monomeric variable antibody domain. Like a whole antibody, it is able to bind selectively to a specific antigen. With a molecular weight of only 12-15 kDa, nanobodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Examples of nanobodies that may be included in the fusion protein include, but are not limited to, anti-Cytotoxic T-Lymphocyte Associated Protein 4 (CTLA4) nanobodies, anti-Programmed death protein 1 (PD-1) nanobodies, anti-Programmed death-ligand 1 (PD-L1) nanobodies, anti-Programmed death-ligand 2 (PD-L2) nanobodies, anti-adenosine A2a receptor (A2AR) nanobodies, anti-B7 homolog 3 protein (B7-H3) nanobodies, anti-B7 homolog 4 protein (B7-H4) nanobodies, anti-B- and T-lymphocyte attenuator (BTLA) nanobodies, anti-killer cell immunoglobulin-like receptor (KIR) nanobodies, anti-Lymphocyte-activation gene 3 (LAG3) nanobodies, anti-T cell immunoglobulin and mucin domain-containing protein 3 (TIM-3) nanobodies, anti-Fibronectin extra type III B module (EIIIB) nanobodies, anti-T cell immunoreceptor with Ig and ITIM domains (TIGIT) nanobodies, anti-Natural killer group 2, member A (NGK2A) nanobodies, anti-P-selectin glycoprotein ligand-1 (PSGL-1) nanobodies, or anti-V-domain immunoglobulin suppressor of T-cell activation (VISTA) nanobodies.

In certain embodiments, the the nanobody comprises an amino acid sequence with at least 75%, 80%, 85%, 90%, 95%, 96%, 97%, 98%, 99% or 100% identity to the amino acid sequence selected from the group consisting of SEQ ID NO: 7-10.

QVQLVETGGGLVQPGGSLRLSCTASGFTFSMHAMTWYRQAPGKQRELVAV
ITSHGDRANYTDSVRGRFTISRDNTKNMVYLQMNSLKPEDTAVYYCNVPR
YDSWGQGTQVTVSSGGLPETGG (PD-L1 binder) (SEQ ID NO:
7)
QVQLVESGGGLVQAGGSLRLSCTASGSTFSRNAMAWFRQAPGKEREFVSG
ISRTGTNSYDADSVKGRFTISKDNAKNTVTLQMNSLKPEDTAIYYCALSQ
TASVATTERLYPYWGQGTQVTVSSGGLPETGG (PD-L1 binder)
(SEQ ID NO: 8)
MAQVQLVESGGGLAQPGGSLRLSCAASGSTISSVAVGWYRQTPGNQREWV
ATSSTSSTTATYADSVKGRFTISRDNAKNTIYLQMNSLKPEDTAVYYCKT
GLTNWGRGTQVTVSSGGLPETGG (CTLA4 binder) (SEQ ID NO:
9)
QVQLVETGGGLVQAGGSLRLSCAASGSTFSHNAGGWYRQAPEKQRELVAG
ISSDGNINYADSVKDRFTISRDNASNTMYLQMNNLKPEDTAVYVCNIRGS
YGNTYYSRWGQGTQVTVSSGGLPETGG (EIIIB binder (SEQ ID
NO: 10; see also SEQ ID NO: 1, 6, 7, 8,
and 9 in WO2019148037).

In further specific embodiments, the fusion proteins comprise an amino acid sequence with at least 75%, 80%, 85%, 90%, 95%, 96%, 97%, 98%, 99% or 100% identity to the amino acid sequence selected from SEQ ID NO: 11-42, wherein residues in parentheses are optional and may be present or may be deleted in whole or in part. The residues in parentheses are signal sequences that may be present or absent, and when present may be substituted with any other signal sequence as deemed appropriate for an intended use.

Based on Mouse STINGΔTM
Anti-PDL1 nanobody - STINGΔTM fusion
(SEQ ID NO: 11)
(MGHHHHHHDYDIPTTENLYFQGSDYKDDDDK)QVQLVETGGGLVQPGGSLRLSCTASGFT
FSMHAMTWYRQAPGKQRELVAVITSHGDRANYTDSVRGRFTISRDNTKNMVYLQMNSLKPE
DTAVYYCNVPRYDSWGQGTQVTVSSGGLPETGGLTPAEVSAVCEEKKLNVAHGLAWSYYIG
YLRLILPGLQARIRMFNQLHNNMLSGAGSRRLYILFPLDCGVPDNLSVVDPNIRFRDMLPQ
QNIDRAGIKNRVYSNSVYEILENGQPAGVCILEYATPLQTLFAMSQDAKAGFSREDRLEQA
KLFCRTLEEILEDVPESRNNCRLIVYQEPTDGNSFSLSQEVLRHIRQEEKEEVTMNAPMTS
VAPPPSVLSQEPRLLISGMDQPLPLRTDLI*
Anti-PDL1 nanobody - STINGΔTM fusion (without optional
residues)
(SEQ ID NO: 12)
QVQLVETGGGLVQPGGSLRLSCTASGFTFSMHAMTWYRQAPGKQRELVAVITSHGDRANYT
DSVRGRFTISRDNTKNMVYLQMNSLKPEDTAVYYCNVPRYDSWGQGTQVTVSSGGLPETGG
LTPAEVSAVCEEKKLNVAHGLAWSYYIGYLRLILPGLQARIRMFNQLHNNMLSGAGSRRLY
ILFPLDCGVPDNLSVVDPNIRFRDMLPQQNIDRAGIKNRVYSNSVYEILENGQPAGVCILE
YATPLQTLFAMSQDAKAGFSREDRLEQAKLFCRTLEEILEDVPESRNNCRLIVYQEPTDGN
SFSLSQEVLRHIRQEEKEEVTMNAPMTSVAPPPSVLSQEPRLLISGMDQPLPLRTDLI*
Anti-PDL1 nanobody - STINGΔTM fusion (new nanobody version)
(SEQ ID NO: 13)
(MGHHHHHHDYDIPTTENLYFQGSDYKDDDDK)QVQLVESGGGLVQAGGSLRLSCTASGST
FSRNAMAWFRQAPGKEREFVSGISRTGTNSYDADSVKGRFTISKDNAKNTVTLQMNSLKPE
DTAIYYCALSQTASVATTERLYPYWGQGTQVTVSSGGLPETGGLTPAEVSAVCEEKKLNVA
HGLAWSYYIGYLRLILPGLQARIRMFNQLHNNMLSGAGSRRLYILFPLDCGVPDNLSVVDP
NIRFRDMLPQQNIDRAGIKNRVYSNSVYEILENGQPAGVCILEYATPLQTLFAMSQDAKAG
FSREDRLEQAKLFCRTLEEILEDVPESRNNCRLIVYQEPTDGNSFSLSQEVLRHIRQEEKE
EVTMNAPMTSVAPPPSVLSQEPRLLISGMDQPLPLRTDLI*
Anti-PDL1 nanobody - STINGΔTM fusion (new nanobody version
minus optional residues)
(SEQ ID NO: 14)
QVQLVESGGGLVQAGGSLRLSCTASGSTFSRNAMAWFRQAPGKEREFVSGISRTGTNSYDA
DSVKGRFTISKDNAKNTVTLQMNSLKPEDTAIYYCALSQTASVATTERLYPYWGQGTQVTV
SSGGLPETGGLTPAEVSAVCEEKKLNVAHGLAWSYYIGYLRLILPGLQARIRMFNQLHNNM
LSGAGSRRLYILFPLDCGVPDNLSVVDPNIRFRDMLPQQNIDRAGIKNRVYSNSVYEILEN
GQPAGVCILEYATPLQTLFAMSQDAKAGFSREDRLEQAKLFCRTLEEILEDVPESRNNCRL
IVYQEPTDGNSFSLSQEVLRHIRQEEKEEVTMNAPMTSVAPPPSVLSQEPRLLISGMDQPL
PLRTDLI*
Anti-CTLA4 nanobody - STINGΔTM fusion
(SEQ ID NO: 15)
(MGHHHHHHDYDIPTTENLYFQGSDYKDDDDK)MAQVQLVESGGGLAQPGGSLRLSCAASG
STISSVAVGWYRQTPGNQREWVATSSTSSTTATYADSVKGRFTISRDNAKNTIYLQMNSLK
PEDTAVYYCKTGLTNWGRGTQVTVSSGGLPETGGLTPAEVSAVCEEKKLNVAHGLAWSYYI
GYLRLILPGLQARIRMFNQLHNNMLSGAGSRRLYILFPLDCGVPDNLSVVDPNIRFRDMLP
QQNIDRAGIKNRVYSNSVYEILENGQPAGVCILEYATPLQTLFAMSQDAKAGFSREDRLEQ
AKLFCRTLEEILEDVPESRNNCRLIVYQEPTDGNSFSLSQEVLRHIRQEEKEEVTMNAPMT
SVAPPPSVLSQEPRLLISGMDQPLPLRTDLI*
Anti-CTLA4 nanobody - STINGΔTM fusion (minus optional
residues)
(SEQ ID NO: 16)
MAQVQLVESGGGLAQPGGSLRLSCAASGSTISSVAVGWYRQTPGNQREWVATSSTSSTTAT
YADSVKGRFTISRDNAKNTIYLQMNSLKPEDTAVYYCKTGLTNWGRGTQVTVSSGGLPETG
GLTPAEVSAVCEEKKLNVAHGLAWSYYIGYLRLILPGLQARIRMFNQLHNNMLSGAGSRRL
YILFPLDCGVPDNLSVVDPNIRFRDMLPQQNIDRAGIKNRVYSNSVYEILENGQPAGVCIL
EYATPLQTLFAMSQDAKAGFSREDRLEQAKLFCRTLEEILEDVPESRNNCRLIVYQEPTDG
NSFSLSQEVLRHIRQEEKEEVTMNAPMTSVAPPPSVLSQEPRLLISGMDQPLPLRTDLI*
Anti- EIIIB nanobody - STINGΔTM fusion
(SEQ ID NO: 17)
(MGHHHHHHDYDIPTTENLYFQGSDYKDDDDK)QVQLVETGGGLVQAGGSLRLSCAASGST
FSHNAGGWYRQAPEKQRELVAGISSDGNINYADSVKDRFTISRDNASNTMYLQMNNLKPED
TAVYVCNIRGSYGNTYYSRWGQGTQVTVSSGGLPETGGLTPAEVSAVCEEKKLNVAHGLAW
SYYIGYLRLILPGLQARIRMFNQLHNNMLSGAGSRRLYILFPLDCGVPDNLSVVDPNIRFR
DMLPQQNIDRAGIKNRVYSNSVYEILENGQPAGVCILEYATPLQTLFAMSQDAKAGFSRED
RLEQAKLFCRTLEEILEDVPESRNNCRLIVYQEPTDGNSFSLSQEVLRHIRQEEKEEVTMN
APMTSVAPPPSVLSQEPRLLISGMDQPLPLRTDLI*
Anti- EIIIB nanobody - STINGΔTM fusion (minus optional
residues)
(SEQ ID NO: 18)
QVQLVETGGGLVQAGGSLRLSCAASGSTFSHNAGGWYRQAPEKQRELVAGISSDGNINYAD
SVKDRFTISRDNASNTMYLQMNNLKPEDTAVYVCNIRGSYGNTYYSRWGQGTQVTVSSGGL
PETGGLTPAEVSAVCEEKKLNVAHGLAWSYYIGYLRLILPGLQARIRMFNQLHNNMLSGAG
SRRLYILFPLDCGVPDNLSVVDPNIRFRDMLPQQNIDRAGIKNRVYSNSVYEILENGQPAG
VCILEYATPLQTLFAMSQDAKAGFSREDRLEQAKLFCRTLEEILEDVPESRNNCRLIVYQE
PTDGNSFSLSQEVLRHIRQEEKEEVTMNAPMTSVAPPPSVLSQEPRLLISGMDQPLPLRTD
LI*
Anti-PDL1 nanobody - STINGΔTM ΔC9 fusion
(SEQ ID NO: 19)
(MGHHHHHHDYDIPTTENLYFQGSDYKDDDDK)QVQLVETGGGLVQPGGSLRLSCTASGFT
FSMHAMTWYROAPGKQRELVAVITSHGDRANYTDSVRGRFTISRDNTKNMVYLQMNSLKPE
DTAVYYCNVPRYDSWGQGTQVTVSSGGLPETGGLTPAEVSAVCEEKKLNVAHGLAWSYYIG
YLRLILPGLQARIRMFNQLHNNMLSGAGSRRLYILFPLDCGVPDNLSVVDPNIRFRDMLPQ
QNIDRAGIKNRVYSNSVYEILENGQPAGVCILEYATPLQTLFAMSQDAKAGFSREDRLEQA
KLFCRTLEEILEDVPESRNNCRLIVYQEPTDGNSFSLSQEVLRHIRQEEKEEVTMNAPMTS
VAPPPSVLSQEPRLLISGMDQ*
Anti-PDL1 nanobody - STINGΔTM ΔC9 fusion (without optional
residues)
(SEQ ID NO: 20)
QVQLVETGGGLVQPGGSLRLSCTASGFTFSMHAMTWYRQAPGKQRELVAVITSHGDRANYT
DSVRGRFTISRDNTKNMVYLQMNSLKPEDTAVYYCNVPRYDSWGQGTQVTVSSGGLPETGG
LTPAEVSAVCEEKKLNVAHGLAWSYYIGYLRLILPGLQARIRMFNQLHNNMLSGAGSRRLY
ILFPLDCGVPDNLSVVDPNIRFRDMLPQQNIDRAGIKNRVYSNSVYEILENGQPAGVCILE
YATPLQTLFAMSQDAKAGFSREDRLEQAKLFCRTLEEILEDVPESRNNCRLIVYQEPTDGN
SFSLSQEVLRHIRQEEKEEVTMNAPMTSVAPPPSVLSQEPRLLISGMDQ*
Anti-PDL1 nanobody - STINGΔTM ΔC9 fusion (new nanobody
version)
(SEQ ID NO: 21)
(MGHHHHHHDYDIPTTENLYFQGSDYKDDDDK)QVQLVESGGGLVQAGGSLRLSCTASGST
FSRNAMAWFRQAPGKEREFVSGISRTGTNSYDADSVKGRFTISKDNAKNTVTLQMNSLKPE
DTAIYYCALSQTASVATTERLYPYWGQGTQVTVSSGGLPETGGLTPAEVSAVCEEKKLNVA
HGLAWSYYIGYLRLILPGLQARIRMFNQLHNNMLSGAGSRRLYILFPLDCGVPDNLSVVDP
NIRFRDMLPQQNIDRAGIKNRVYSNSVYEILENGQPAGVCILEYATPLQTLFAMSQDAKAG
FSREDRLEQAKLFCRTLEEILEDVPESRNNCRLIVYQEPTDGNSFSLSQEVLRHIRQEEKE
EVTMNAPMTSVAPPPSVLSQEPRLLISGMDQ*
Anti-PDL1 nanobody - STINGΔTM ΔC9 fusion (new nanobody
version minus optional residues)
(SEQ ID NO: 22)
QVQLVESGGGLVQAGGSLRLSCTASGSTFSRNAMAWFRQAPGKEREFVSGISRTGTNSYDA
DSVKGRFTISKDNAKNTVTLQMNSLKPEDTAIYYCALSQTASVATTERLYPYWGQGTQVTV
SSGGLPETGGLTPAEVSAVCEEKKLNVAHGLAWSYYIGYLRLILPGLQARIRMFNQLHNNM
LSGAGSRRLYILFPLDCGVPDNLSVVDPNIRFRDMLPQQNIDRAGIKNRVYSNSVYEILEN
GQPAGVCILEYATPLQTLFAMSQDAKAGFSREDRLEQAKLFCRTLEEILEDVPESRNNCRL
IVYQEPTDGNSFSLSQEVLRHIRQEEKEEVTMNAPMTSVAPPPSVLSQEPRLLISGMDQ*
Anti-CTLA4 nanobody - STINGΔTM ΔC9 fusion
(SEQ ID NO: 23)
(MGHHHHHHDYDIPTTENLYFQGSDYKDDDDK)MAQVQLVESGGGLAQPGGSLRLSCAASG
STISSVAVGWYRQTPGNQREWVATSSTSSTTATYADSVKGRFTISRDNAKNTIYLQMNSLK
PEDTAVYYCKTGLTNWGRGTQVTVSSGGLPETGGLTPAEVSAVCEEKKLNVAHGLAWSYYI
GYLRLILPGLQARIRMFNQLHNNMLSGAGSRRLYILFPLDCGVPDNLSVVDPNIRFRDMLP
QQNIDRAGIKNRVYSNSVYEILENGQPAGVCILEYATPLQTLFAMSQDAKAGFSREDRLEQ
AKLFCRTLEEILEDVPESRNNCRLIVYQEPTDGNSFSLSQEVLRHIRQEEKEEVTMNAPMT
SVAPPPSVLSQEPRLLISGMDQ*
Anti-CTLA4 nanobody - STINGΔTM ΔC9 fusion (minus optional
residues)
(SEQ ID NO: 24)
MAQVQLVESGGGLAQPGGSLRLSCAASGSTISSVAVGWYRQTPGNQREWVATSSTSSTTAT
YADSVKGRFTISRDNAKNTIYLQMNSLKPEDTAVYYCKTGLTNWGRGTQVTVSSGGLPETG
GLTPAEVSAVCEEKKLNVAHGLAWSYYIGYLRLILPGLQARIRMFNQLHNNMLSGAGSRRL
YILFPLDCGVPDNLSVVDPNIRFRDMLPQQNIDRAGIKNRVYSNSVYEILENGQPAGVCIL
EYATPLQTLFAMSQDAKAGFSREDRLEQAKLFCRTLEEILEDVPESRNNCRLIVYQEPTDG
NSFSLSQEVLRHIRQEEKEEVTMNAPMTSVAPPPSVLSQEPRLLISGMDQ*
Anti- EIIIB nanobody - STINGΔTM ΔC9 fusion
(SEQ ID NO: 25)
(MGHHHHHHDYDIPTTENLYFQGSDYKDDDDK)QVQLVETGGGLVQAGGSLRLSCAASGST
FSHNAGGWYRQAPEKQRELVAGISSDGNINYADSVKDRFTISRDNASNTMYLQMNNLKPED
TAVYVCNIRGSYGNTYYSRWGQGTQVTVSSGGLPETGGLTPAEVSAVCEEKKLNVAHGLAW
SYYIGYLRLILPGLQARIRMFNQLHNNMLSGAGSRRLYILFPLDCGVPDNLSVVDPNIRFR
DMLPQQNIDRAGIKNRVYSNSVYEILENGQPAGVCILEYATPLQTLFAMSQDAKAGFSRED
RLEQAKLFCRTLEEILEDVPESRNNCRLIVYQEPTDGNSFSLSQEVLRHIRQEEKEEVTMN
APMTSVAPPPSVLSQEPRLLISGMDQ*
Anti- EIIIB nanobody - STINGΔTM ΔC9 fusion (minus optional
residues)
(SEQ ID NO: 26)
QVQLVETGGGLVQAGGSLRLSCAASGSTFSHNAGGWYRQAPEKQRELVAGISSDGNINYAD
SVKDRFTISRDNASNTMYLQMNNLKPEDTAVYVCNIRGSYGNTYYSRWGQGTQVTVSSGGL
PETGGLTPAEVSAVCEEKKLNVAHGLAWSYYIGYLRLILPGLQARIRMFNQLHNNMLSGAG
SRRLYILFPLDCGVPDNLSVVDPNIRFRDMLPQQNIDRAGIKNRVYSNSVYEILENGQPAG
VCILEYATPLQTLFAMSQDAKAGFSREDRLEQAKLFCRTLEEILEDVPESRNNCRLIVYQE
PTDGNSFSLSQEVLRHIRQEEKEEVTMNAPMTSVAPPPSVLSQEPRLLISGMDQ*
Based on Human STINGΔTM
Anti-PDL1 nanobody - STINGΔTM fusion
(SEQ ID NO: 27)
(MGHHHHHHDYDIPTTENLYFQGSDYKDDDDK)QVQLVETGGGLVQPGGSLRLSCTASGFT
FSMHAMTWYRQAPGKQRELVAVITSHGDRANYTDSVRGRFTISRDNTKNMVYLQMNSLKPE
DTAVYYCNVPRYDSWGQGTQVTVSSGGLPETGGLAPAEISAVCEKGNFNVAHGLAWSYYIG
YLRLILPELQARIRTYNQHYNNLLRGAVSQRLYILLPLDCGVPDNLSMADPNIRFLDKLPQ
QTGDRAGIKDRVYSNSIYELLENGQRAGTCVLEYATPLQTLFAMSQYSQAGFSREDRLEQA
KLFCRTLEDILADAPESQNNCRLIAYQEPADDSSFSLSQEVLRHLRQEEKEEVTVGSLKTS
AVPSTSTMSQEPELLISGMEKPLPLRTDFS*
Anti-PDL1 nanobody - STINGΔTM fusion (without optional
residues)
(SEQ ID NO: 28)
QVQLVETGGGLVQPGGSLRLSCTASGFTFSMHAMTWYRQAPGKQRELVAVITSHGDRANYT
DSVRGRFTISRDNTKNMVYLQMNSLKPEDTAVYYCNVPRYDSWGQGTQVTVSSGGLPETGG
LAPAEISAVCEKGNFNVAHGLAWSYYIGYLRLILPELQARIRTYNQHYNNLLRGAVSQRLY
ILLPLDCGVPDNLSMADPNIRFLDKLPQQTGDRAGIKDRVYSNSIYELLENGQRAGTCVLE
YATPLQTLFAMSQYSQAGFSREDRLEQAKLFCRTLEDILADAPESQNNCRLIAYQEPADDS
SFSLSQEVLRHLRQEEKEEVTVGSLKTSAVPSTSTMSQEPELLISGMEKPLPLRTDFS*
Anti-PDL1 nanobody - STINGΔTM fusion (new nanobody version)
(SEQ ID NO: 29)
(MGHHHHHHDYDIPTTENLYFQGSDYKDDDDK)QVQLVESGGGLVQAGGSLRLSCTASGST
FSRNAMAWFRQAPGKEREFVSGISRTGTNSYDADSVKGRFTISKDNAKNTVTLQMNSLKPE
DTAIYYCALSQTASVATTERLYPYWGQGTQVTVSSGGLPETGGLAPAEISAVCEKGNFNVA
HGLAWSYYIGYLRLILPELQARIRTYNQHYNNLLRGAVSQRLYILLPLDCGVPDNLSMADP
NIRFLDKLPQQTGDRAGIKDRVYSNSIYELLENGQRAGTCVLEYATPLQTLFAMSQYSQAG
FSREDRLEQAKLFCRTLEDILADAPESQNNCRLIAYQEPADDSSFSLSQEVLRHLRQEEKE
EVTVGSLKTSAVPSTSTMSQEPELLISGMEKPLPLRTDFS*
Anti-PDL1 nanobody - STINGΔTM fusion (new nanobody version
minus optional residues)
(SEQ ID NO: 30)
QVQLVESGGGLVQAGGSLRLSCTASGSTFSRNAMAWFRQAPGKEREFVSGISRTGTNSYDA
DSVKGRFTISKDNAKNTVTLQMNSLKPEDTAIYYCALSQTASVATTERLYPYWGQGTQVTV
SSGGLPETGGLAPAEISAVCEKGNFNVAHGLAWSYYIGYLRLILPELQARIRTYNQHYNNL
LRGAVSQRLYILLPLDCGVPDNLSMADPNIRFLDKLPQQTGDRAGIKDRVYSNSIYELLEN
GQRAGTCVLEYATPLQTLFAMSQYSQAGFSREDRLEQAKLFCRTLEDILADAPESQNNCRL
IAYQEPADDSSFSLSQEVLRHLRQEEKEEVTVGSLKTSAVPSTSTMSQEPELLISGMEKPL
PLRTDFS*
Anti-CTLA4 nanobody - STINGΔTM fusion
(SEQ ID NO: 31)
(MGHHHHHHDYDIPTTENLYFQGSDYKDDDDK)MAQVQLVESGGGLAQPGGSLRLSCAASG
STISSVAVGWYRQTPGNQREWVATSSTSSTTATYADSVKGRFTISRDNAKNTIYLQMNSLK
PEDTAVYYCKTGLTNWGRGTQVTVSSGGLPETGGLAPAEISAVCEKGNFNVAHGLAWSYYI
GYLRLILPELQARIRTYNQHYNNLLRGAVSQRLYILLPLDCGVPDNLSMADPNIRFLDKLP
QQTGDRAGIKDRVYSNSIYELLENGQRAGTCVLEYATPLQTLFAMSQYSQAGFSREDRLEQ
AKLFCRTLEDILADAPESQNNCRLIAYQEPADDSSFSLSQEVLRHLRQEEKEEVTVGSLKT
SAVPSTSTMSQEPELLISGMEKPLPLRTDFS*
Anti-CTLA4 nanobody - STINGΔTM fusion (minus optional
residues)
(SEQ ID NO: 32)
MAQVQLVESGGGLAQPGGSLRLSCAASGSTISSVAVGWYRQTPGNQREWVATSSTSSTTAT
YADSVKGRFTISRDNAKNTIYLQMNSLKPEDTAVYYCKTGLTNWGRGTQVTVSSGGLPETG
GLAPAEISAVCEKGNFNVAHGLAWSYYIGYLRLILPELQARIRTYNQHYNNLLRGAVSQRL
YILLPLDCGVPDNLSMADPNIRFLDKLPQQTGDRAGIKDRVYSNSIYELLENGQRAGTCVL
EYATPLQTLFAMSQYSQAGFSREDRLEQAKLFCRTLEDILADAPESQNNCRLIAYQEPADD
SSFSLSQEVLRHLRQEEKEEVTVGSLKTSAVPSTSTMSQEPELLISGMEKPLPLRTDFS*
Anti- EIIIB nanobody - STINGΔTM fusion
(SEQ ID NO: 33)
(MGHHHHHHDYDIPTTENLYFQGSDYKDDDDK)QVQLVETGGGLVQAGGSLRLSCAASGST
FSHNAGGWYRQAPEKQRELVAGISSDGNINYADSVKDRFTISRDNASNTMYLQMNNLKPED
TAVYVCNIRGSYGNTYYSRWGQGTQVTVSSGGLPETGGLAPAEISAVCEKGNFNVAHGLAW
SYYIGYLRLILPELQARIRTYNQHYNNLLRGAVSQRLYILLPLDCGVPDNLSMADPNIRFL
DKLPQQTGDRAGIKDRVYSNSIYELLENGQRAGTCVLEYATPLQTLFAMSQYSQAGFSRED
RLEQAKLFCRTLEDILADAPESQNNCRLIAYQEPADDSSFSLSQEVLRHLRQEEKEEVTVG
SLKTSAVPSTSTMSQEPELLISGMEKPLPLRTDF*
Anti- EIIIB nanobody - STINGΔTM fusion (minus optional
residues)
(SEQ ID NO: 34)
QVQLVETGGGLVQAGGSLRLSCAASGSTFSHNAGGWYRQAPEKQRELVAGISSDGNINYAD
SVKDRFTISRDNASNTMYLQMNNLKPEDTAVYVCNIRGSYGNTYYSRWGQGTQVTVSSGGL
PETGGLAPAEISAVCEKGNFNVAHGLAWSYYIGYLRLILPELQARIRTYNQHYNNLLRGAV
SQRLYILLPLDCGVPDNLSMADPNIRFLDKLPQQTGDRAGIKDRVYSNSIYELLENGQRAG
TCVLEYATPLQTLFAMSQYSQAGFSREDRLEQAKLFCRTLEDILADAPESQNNCRLIAYQE
PADDSSFSLSQEVLRHLRQEEKEEVTVGSLKTSAVPSTSTMSQEPELLISGMEKPLPLRTD
F*
Anti-PDL1 nanobody - STINGΔTM ΔC9 fusion
(SEQ ID NO: 35)
(MGHHHHHHDYDIPTTENLYFQGSDYKDDDDK)QVQLVETGGGLVQPGGSLRLSCTASGFT
FSMHAMTWYRQAPGKQRELVAVITSHGDRANYTDSVRGRFTISRDNTKNMVYLQMNSLKPE
DTAVYYCNVPRYDSWGQGTQVTVSSGGLPETGGLAPAEISAVCEKGNFNVAHGLAWSYYIG
YLRLILPELQARIRTYNQHYNNLLRGAVSQRLYILLPLDCGVPDNLSMADPNIRFLDKLPQ
QTGDRAGIKDRVYSNSIYELLENGQRAGTCVLEYATPLQTLFAMSQYSQAGFSREDRLEQA
KLFCRTLEDILADAPESQNNCRLIAYQEPADDSSFSLSQEVLRHLRQEEKEEVTVGSLKTS
AVPSTSTMSQEPELLISGMEK*
Anti-PDL1 nanobody - STINGΔTM ΔC9 fusion (without optional
residues)
(SEQ ID NO: 36)
QVQLVETGGGLVQPGGSLRLSCTASGFTFSMHAMTWYRQAPGKQRELVAVITSHGDRANYT
DSVRGRFTISRDNTKNMVYLQMNSLKPEDTAVYYCNVPRYDSWGQGTQVTVSSGGLPETGG
LAPAEISAVCEKGNFNVAHGLAWSYYIGYLRLILPELQARIRTYNQHYNNLLRGAVSQRLY
ILLPLDCGVPDNLSMADPNIRFLDKLPQQTGDRAGIKDRVYSNSIYELLENGQRAGTCVLE
YATPLQTLFAMSQYSQAGFSREDRLEQAKLFCRTLEDILADAPESQNNCRLIAYQEPADDS
SESLSQEVLRHLRQEEKEEVTVGSLKTSAVPSTSTMSQEPELLISGMEK*
Anti-PDL1 nanobody - STINGΔTM ΔC9 fusion (new nanobody
version)
(SEQ ID NO: 37)
(MGHHHHHHDYDIPTTENLYFQGSDYKDDDDK)QVQLVESGGGLVQAGGSLRLSCTASGST
FSRNAMAWFRQAPGKEREFVSGISRTGTNSYDADSVKGRFTISKDNAKNTVTLQMNSLKPE
DTAIYYCALSQTASVATTERLYPYWGQGTQVTVSSGGLPETGGLAPAEISAVCEKGNFNVA
HGLAWSYYIGYLRLILPELQARIRTYNQHYNNLLRGAVSQRLYILLPLDCGVPDNLSMADP
NIRFLDKLPQQTGDRAGIKDRVYSNSIYELLENGQRAGTCVLEYATPLQTLFAMSQYSQAG
FSREDRLEQAKLFCRTLEDILADAPESQNNCRLIAYQEPADDSSFSLSQEVLRHLRQEEKE
EVTVGSLKTSAVPSTSTMSQEPELLISGMEK*
Anti-PDL1 nanobody - STINGΔTM ΔC9 fusion (new nanobody
version minus optional residues)
(SEQ ID NO: 38)
QVQLVESGGGLVQAGGSLRLSCTASGSTFSRNAMAWFRQAPGKEREFVSGISRTGTNSYDA
DSVKGRFTISKDNAKNTVTLQMNSLKPEDTAIYYCALSQTASVATTERLYPYWGQGTQVTV
SSGGLPETGGLAPAEISAVCEKGNFNVAHGLAWSYYIGYLRLILPELQARIRTYNQHYNNL
LRGAVSQRLYILLPLDCGVPDNLSMADPNIRFLDKLPQQTGDRAGIKDRVYSNSIYELLEN
GQRAGTCVLEYATPLQTLFAMSQYSQAGFSREDRLEQAKLFCRTLEDILADAPESQNNCRL
IAYQEPADDSSFSLSQEVLRHLRQEEKEEVTVGSLKTSAVPSTSTMSQEPELLISGMEK*
Anti-CTLA4 nanobody - STINGΔTM ΔC9 fusion
(SEQ ID NO: 39)
(MGHHHHHHDYDIPTTENLYFQGSDYKDDDDK)MAQVQLVESGGGLAQPGGSLRLSCAASG
STISSVAVGWYRQTPGNQREWVATSSTSSTTATYADSVKGRFTISRDNAKNTIYLQMNSLK
PEDTAVYYCKTGLTNWGRGTQVTVSSGGLPETGGLAPAEISAVCEKGNFNVAHGLAWSYYI
GYLRLILPELQARIRTYNQHYNNLLRGAVSQRLYILLPLDCGVPDNLSMADPNIRFLDKLP
QQTGDRAGIKDRVYSNSIYELLENGQRAGTCVLEYATPLQTLFAMSQYSQAGFSREDRLEQ
AKLFCRTLEDILADAPESQNNCRLIAYQEPADDSSFSLSQEVLRHLRQEEKEEVTVGSLKT
SAVPSTSTMSQEPELLISGMEK*
Anti-CTLA4 nanobody - STINGΔTM ΔC9 fusion (minus optional
residues)
(SEQ ID NO: 40)
MAQVQLVESGGGLAQPGGSLRLSCAASGSTISSVAVGWYRQTPGNQREWVATSSTSSTTAT
YADSVKGRFTISRDNAKNTIYLQMNSLKPEDTAVYYCKTGLTNWGRGTQVTVSSGGLPETG
GLAPAEISAVCEKGNFNVAHGLAWSYYIGYLRLILPELQARIRTYNQHYNNLLRGAVSQRL
YILLPLDCGVPDNLSMADPNIRFLDKLPQQTGDRAGIKDRVYSNSIYELLENGQRAGTCVL
EYATPLQTLFAMSQYSQAGFSREDRLEQAKLFCRTLEDILADAPESQNNCRLIAYQEPADD
SSESLSQEVLRHLRQEEKEEVTVGSLKTSAVPSTSTMSQEPELLISGMEK*
Anti- EIIIB nanobody - STINGΔTM ΔC9 fusion
(SEQ ID NO: 41)
(MGHHHHHHDYDIPTTENLYFQGSDYKDDDDK)QVQLVETGGGLVQAGGSLRLSCAASGST
FSHNAGGWYRQAPEKQRELVAGISSDGNINYADSVKDRFTISRDNASNTMYLQMNNLKPED
TAVYVCNIRGSYGNTYYSRWGQGTQVTVSSGGLPETGGLAPAEISAVCEKGNFNVAHGLAW
SYYIGYLRLILPELQARIRTYNQHYNNLLRGAVSQRLYILLPLDCGVPDNLSMADPNIRFL
DKLPQQTGDRAGIKDRVYSNSIYELLENGQRAGTCVLEYATPLQTLFAMSQYSQAGFSRED
RLEQAKLFCRTLEDILADAPESQNNCRLIAYQEPADDSSFSLSQEVLRHLRQEEKEEVTVG
SLKTSAVPSTSTMSQEPELLISGMEK*
Anti- EIIIB nanobody - STINGΔTM ΔC9 fusion (minus optional
residues)
(SEQ ID NO: 42)
QVQLVETGGGLVQAGGSLRLSCAASGSTFSHNAGGWYRQAPEKQRELVAGISSDGNINYAD
SVKDRFTISRDNASNTMYLQMNNLKPEDTAVYVCNIRGSYGNTYYSRWGQGTQVTVSSGGL
PETGGLAPAEISAVCEKGNFNVAHGLAWSYYIGYLRLILPELQARIRTYNQHYNNLLRGAV
SQRLYILLPLDCGVPDNLSMADPNIRFLDKLPQQTGDRAGIKDRVYSNSIYELLENGQRAG
TCVLEYATPLQTLFAMSQYSQAGFSREDRLEQAKLFCRTLEDILADAPESQNNCRLIAYQE
PADDSSFSLSQEVLRHLRQEEKEEVTVGSLKTSAVPSTSTMSQEPELLISGMEK*

In further embodiments, the fusion proteins of the disclosure may further comprise a cell penetrating peptide, as disclosed herein.

In another aspect, the disclosure provides nucleic acids encoding a fusion protein of the disclosure. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In another aspect, disclosure provides expression vectors comprising the nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence is still considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In one aspect, the present disclosure provides cells comprising the fusion protein, the nucleic acid, and/or the expression vector of any embodiment or combination of embodiments of the disclosure, wherein the cells can be either prokaryotic or eukaryotic, such as mammalian cells. In one embodiment, the cells may be transiently or stably transfected with the nucleic acids or expression vectors of the disclosure. Such transfection of expression vectors into prokaryotic and eukaryotic cells can be accomplished via any technique known in the art. A method of producing a fusion protein according to the invention is an additional part of the invention. The method comprises the steps of (a) culturing a host according to this aspect of the invention under conditions conducive to the expression of the fusion protein, and (b) optionally, recovering the expressed fusion protein.

The host cells can be fungal cells including yeast such as Saccharomyces cerevisiae, Pichia pastoris, or Schizosaccharomyces pombe. The host cells also be any of various animal cells, such as insect cells (e.g., Sf-9) or mammalian cells (e.g., HEK293F, CHO, COS-7, NIH-3T3, NS0, PER.C6®, and hybridoma). In further embodiments, the host cells is a CHO cell selected from CHO-K, CHO-0, CHO-Lec10, CHO-Lec13, CHO-Lec1, CHO Pro-5, and CHO dhfr⁻. In particular embodiments, the host cell is a hybridoma.

In one aspect, the disclosure provides compositions, comprising the fusion protein of any embodment or combination of embodiments herein and a STING agonist. The compositins may be used, for example, in the therapeutic methods of the disclosure.

Examples of STING agonists include, but are not limited to:
(1) Natural and synthetic CDNs as direct STING agonists: c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP.
(2) Non-nucleotidyl small molecule STING agonists: 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo [3,2-a:3′,2′-d]pyrazine-5,10(5aH,10aH)-dione, 4-(2-chloro-6-fluorobenzyl)-N-(furan-2-yl methyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3 -b][1,4]thiazine-7-carboxamide, 2-oxo-1,2,3,4-tetrahydroquinoline-7-carboxamide, or 2-Oxo-1,2,3,4-tetrahydroquinazoline-7-carboxamides.

In one embodiment, the STING agonist is a cytosolic cyclic dinucleotide (CDN). In another embodiment, the CDN is c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP.

In another embodiment, the STING agonist is a non-nucleotidyl small molecule. In exemplary embodiments, the non-nucleotidyl small molecule is 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo[3,2-a: 3′,2′-d]pyrazine-5,10(5aH,10aH)-dione, 4-(2-chloro-6-fluorobenzyl)-N-(furan-2-ylmethyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3-b][1,4]thiazine-7-carboxamide, 2-oxo-1,2,3,4-tetrahydroquinoline-7-carboxamide, or 2-Oxo-1,2,3,4-tetrahydroquinazoline-7-carboxamides.

In another aspect, the present disclosure provides methods of treating cancer or an infectious disease, comprising administering the composition of any embodiment or combination of embodiments of the disclosure.

The cytosolic DNA sensing pathway involving cyclic GMP-AMP synthase (cGAS) and the stimulator of interferon genes (STING) represents an essential innate immune mechanism in response to foreign pathogens. Upon detection of cytosolic DNA, the intracellular nucleic acid sensor cGAS catalyzes the productions of cyclic dinucleotides (CDNs) such as 2′3′-cyclic GMP-AMP (cGAMP), which functions as a second messenger to bind the adaptor protein

STING to initiate type I interferon (IFN) production and boost dendritic cell (DC) maturation and T cell infiltration. Meanwhile, the cGAS-STING signaling pathway is profound at sensing neoplastic progression by promoting type I IFN production and initiating cytotoxic T cell-mediated anti-tumor immune response. Synthetic STING agonists can be utilized to activate the innate and adaptive immune responses as a monotherapy or in combination with immune checkpoint blockade (ICB) for cancer immunotherapy.

As disclosed in the examples that follow, compositions of the disclosure, exemplified using cGAMP as the STING agonist, eliminated subcutaneous MC38 and YUMM1.7 tumors in 70%-100% of mice and protected all cured mice against rechallenge. Mechanistic studies revealed a robust T_{H}1 polarization and suppression of Treg of CD4⁺ T cells, followed by an effective collaboration of CD4⁺ T, CD8⁺ T and NK cells to eliminate tumors. The examples further demonstrate the potential to overcome host STING deficiency by significantly decreasing MC38 tumor burden in STING-KO mice, addressing the translational challenge for the 19% of human population with loss-of-function STING variants. The cytosolic DNA sensing pathway involving cyclic GMP-AMP synthase (cGAS) and the stimulator of interferon genes (STING) represents an essential innate immune mechanism in response to foreign pathogens. Upon detection of cytosolic DNA, the intracellular nucleic acid sensor cGAS catalyzes the productions of cyclic dinucleotides (CDNs) such as 2′3′-cyclic GMP-AMP (cGAMP), which functions as a second messenger to bind the adaptor protein STING to initiate type I interferon (IFN) production and boost dendritic cell (DC) maturation and T cell infiltration. Meanwhile, the cGAS-STING signaling pathway is profound at sensing neoplastic progression by promoting type I IFN production and initiating cytotoxic T cell-mediated anti-tumor immune response. Synthetic STING agonists can be utilized to activate the innate and adaptive immune responses as a monotherapy or in combination with immune checkpoint blockade (ICB) for cancer immunotherapy.

### Definition

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The terms “a,” “an” and “the” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

The term “comprise” is generally used in the sense of include, that is to say permitting the presence of one or more features or components. Wherever embodiments, are described herein with the language “comprising,” otherwise analogous embodiments described in terms of “consisting of,” and/or “consisting essentially of” are also provided.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In one embodiment, the administering is done intra-tumorally.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The term “a small molecule” is a compound having a molecular weight of less than 2000 Daltons, preferably less than 1000 Daltons. Typically, a small molecule therapeutic is an organic compound that may help regulate a biological process.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer. The skilled worker can readily determine the effective amount for a given situation by routine experimentation.

The terms “cancer,” “tumor,” “cancerous,” and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al., Br. J. Cancer 94:1057-1065 (2006)), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer and cancers of mucinous origins, such as mucinous ovarian cancer, cholangiocarcinoma (liver) and renal papillary carcinoma. In particular embodiments, the cancer is breast, endometrial, or uterine cancer. In other embodiments, the cancer is a myeloma (e.g., multiple myeloma, plasmacytoma, localized myeloma, and extramedullary myeloma), or endometrial, gastric, liver, colon, renal or pancreatic cancer. In some embodiments, the cancer comprises a colon carcinoma, a melanoma, or breast cancer.

A “recombinant” polypeptide, protein or antibody refers to polypeptide, protein or antibody produced via recombinant DNA technology. Recombinantly produced polypeptides, proteins and antibodies expressed in host cells are considered isolated for the purpose of the present disclosure, as are native or recombinant polypeptides which have been separated, fractionated, or partially or substantially purified by any suitable technique.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site. Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI).

### Cell-Penetrating Peptides

The term “cell-penetrating-peptide sequence” is used in the present specification interchangeably with “CPP”, “protein transducing domain” or “PTD”. It refers to a peptide chain of variable length that directs the transport of a protein inside a cell. The delivering process into cell commonly occurs by endocytosis but the peptide can also be internalized into cell by means of direct membrane translocation. CPPs typically have an amino acid composition that either contains a high relative abundance of positively charged amino acids such as lysine or arginine or has sequences that contain an alternating pattern of polar/charged amino acid and non-polar, hydrophobic amino acids.

Cell-penetrating peptides (CPPs) are short peptides that facilitate cellular intake and uptake of molecules ranging from nanosize particles to small chemical compounds to large fragments of DNA. The “cargo” is associated with the peptides either through chemical linkage via covalent bonds or through non-covalent interactions. CPPs deliver the cargo into cells, commonly through endocytosis.

CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake. Numerous CPPs are known in the art, any of which can be part of the heterologous fusion proteins of the present invention.

### Preparation of Fusion Proteins Comprising STINGΔTM Protein Fused to a Cell-Penetrating Domain or a Nanobody

The fusion proteins comprising STINGΔTM protein fused to a cell-penetrating domain or a nanobody of the compositions may be produced by either synthetic chemical processes or by recombinant methods or a combination of both methods. The fusion proteins comprising STINGΔTM protein fused to a cell-penetrating domain or a nanobody may be prepared as full-length polymers or be synthesized as non-full length fragments and joined. Chemical synthesis of peptides is routinely performed by methods well known to those skilled in the art for either solid phase or solution phase peptide synthesis. For solid phase peptide synthesis, so called t-Boc (tert-Butyloxy carbonyl) and Fmoc (Fluorenyl-methoxy-carbonyl) chemistry, referring to the N-terminal protecting groups, on polyamide or polystyrene resin have become the conventional methods (Merrifield, R B. 1963 and Sheppard, R C. 1971, respectively). Unlike ribosomal protein synthesis, solid-phase peptide synthesis proceeds in a C-terminal to N-terminal fashion. The N-termini of amino acid monomers is protected by these two groups and added onto a deprotected amino acid chain. Deprotection requires strong acid such as trifluoroacetic acid for t-Boc and bases such as piperidine for Fmoc. Stepwise elongation, in which the amino acids are connected step-by-step in turn, is ideal for small peptides containing between 2 and 100 amino acid residues.

Non-naturally occurring residues may be incorporated into fusion proteins comprising STINGΔTM protein fused to a cell-penetrating domain or a nanobody. Examples of non-ribosomally installed amino acids that may be used in accordance with a present invention and still form a peptide backbone include, but are not limited to: D-amino acids, β-amino acids, pseudo-glutamate, γ-aminobutyrate, ornithine, homocysteine, N-substituted amino acids (R. Simon et al., Proc. Natl. Acad. Sci. U.S.A. (1992) 89: 9367-71; WO 91/19735 (Bartlett et al.; incorporated by reference), U.S. Pat. No. 5,646,285 (Baindur; incorporated by reference), α-aminomethyleneoxy acetic acids (an amino acid-Gly dipeptide isostere), and α-aminooxy acids and other amino acid derivatives having non-genetically non-encoded side chain function groups etc. Peptide analogs containing thioamide, vinylogous amide, hydrazino, methyleneoxy, thiomethylene, phosphonamides, oxyamide, hydroxyethylene, reduced amide and substituted reduced amide isosteres and β-sulfonamide(s) may be employed.

In another process, unnatural amino acids have been introduced into recombinantly produced proteins by a method of codon suppression. In one aspect, the use of codon suppression techniques could be adapted to introduce an aldehyde or ketone functional group or any other functional group in any suitable position within a polypeptide chain for conjugation (see e.g. WO 2006/132969; incorporated by reference).

Alternatively, recombinant expression methods are particularly useful. Recombinant protein expression using a host cell (a cell artificially engineered to comprise nucleic acids encoding the sequence of the peptide and which will transcribe and translate, and, optionally, secrete the peptide into the cell growth medium) is used routinely in the art. For recombinant production process, a nucleic acid coding for the amino acid sequence of the peptide would typically be synthesized by conventional methods and integrated into an expression vector. Such methods are particularly preferred for manufacture of the polypeptide compositions comprising the peptides fused to additional polypeptide sequences or other proteins or protein fragments or domains. The host cell can optionally be at least one selected from E. coli, COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Also provided is a method for producing at least one peptide, comprising translating the peptide encoding nucleic acid under conditions in vitro, in vivo or in situ, such that the peptide is expressed in detectable or recoverable amounts. The techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989).

Methods of fusing antibodies like nanobodies with proteins is known in the art, see, e.g., LaFleur, et al., MAbs. 2013 March-April;5(2):208-18. Small binding domains can be fused to multiple locations on antibodies and still retain binding affinity to ligand and antigen.

### Pharmaceutical Compositions and Administration Methods

Methods of preparing and administering a composition comprising a fusion protein and a STING agonist, wherein the fusion protein comprises STINGΔTM protein fused to a cell-penetrating domain or a nanobody. The methods of administering the composition to a subject in need thereof are known to or are readily determined by those of ordinary skill in the art. The route of administration of the composition can be, for example, oral, parenteral, by inhalation or topical. The term parenteral includes, e.g., intravenous, intraarterial, intraperitoneal, intramuscular, intraocular, subcutaneous, rectal, or vaginal administration. While all these forms of administration are clearly contemplated as being within the scope of the disclosure, another example of a form for administration would be a solution for injection, in particular for intravenous or intraarterial injection or drip. Usually, a suitable pharmaceutical composition can comprise a buffer (e.g., acetate, phosphate or citrate buffer), a surfactant (e.g., polysorbate), optionally a stabilizer agent (e.g., human albumin), etc. In other methods compatible with the teachings herein, the composition as provided herein can be delivered directly to the organ and/or site of a fibrosis or tumor, thereby increasing the exposure of the diseased tissue to therapeutic agent.

As discussed herein, the composition can be administered in a pharmaceutically effective amount for the in vivo treatment of cancer. In this regard, it will be appreciated that the disclosed composition can be formulated so as to facilitate administration and promote stability of the active agent. Pharmaceutical compositions in accordance with the disclosure can comprise a pharmaceutically acceptable, non-toxic, sterile carrier such as physiological saline, non-toxic buffers, preservatives and the like. Suitable formulations for use in therapeutic methods disclosed herein are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions or solutions. Such compositions can be prepared as solutions in saline, employing benzyl alcohol or other suitable preservatives, absorption promoters to enhance bioavailability, and/or other conventional solubilizing or dispersing agents.

### Methods of Use and Pharmaceutical Compositions

The provided compositions comprising a fusion protein and a STING agonist, wherein the fusion protein comprises STINGΔTM protein fused to a cell-penetrating domain or a nanobody are useful in a variety of applications including, but not limited to, methods of treating and/or ameliorating various diseases and conditions. Methods are provided for the use of the disclosed compositions to treat subjects having a disease or condition associated with STING signaling, altered STING expression. The composition disclosed herein may be used to treat auto-inflammation, virus infection or cancers.

In certain embodiments, the disclosure provides a method of treating cancer that comprises contacting a cancer cell, tumor associated-stromal cell, tumor extracellular matrices, or endothelial cell with the disclosed composition. In additional embodiments, the cancer cell is a myeloma (e.g., multiple myeloma, plasmacytoma, localized myeloma, or extramedullary myeloma), ovarian, breast, colon, endometrial, liver, kidney, pancreatic, gastric, uterine and/or colon cancer cell. In some embodiments, the contacted cell is from a cancer line. In some embodiments, the cancer cell is contacted in vivo.

### Combination Therapies

In some embodiments, the composition comprising a fusion protein and a STING agonist, wherein the fusion protein comprises STINGΔTM protein fused to a cell-penetrating domain or a nanobody is administered alone or as a combination therapy. In some embodiments, composition is administered in combination with one or more other therapies. Such therapies include additional therapeutic agents as well as other medical interventions. Exemplary therapeutic agents that can be administered in combination with the composition provided herein include, but are not limited to, chemotherapeutic agents, and/or immunomodulators. In various embodiments, the composition is administered to a subject before, during, and/or after a surgical excision/removal procedure.

### EXAMPLES

Stimulator of interferon genes (STING) signaling is a promising target in cancer immunotherapy. Here, we report a distinct CD4-mediated, protein-based combination therapy of STING and ICB as an in situ vaccine. The treatment eliminated subcutaneous MC38 and YUMM1.7 tumors in 70%-100% of mice and protected all cured mice against rechallenge. Mechanistic studies revealed a robust TH1 polarization and suppression of Treg of CD4⁺ T cells, followed by an effective collaboration of CD4⁺ T, CD8⁺ T and NK cells to eliminate tumors. Finally, we demonstrated the potential to overcome host STING deficiency by significantly decreasing MC38 tumor burden in STING-KO mice, addressing the translational challenge for the 19% of human population with loss-of-function STING variants.

### Introduction

Immune checkpoint blockade (ICB) has revolutionized the landscape of cancer immunotherapy over the past decades as one of the most promising treatment options for diseases such as advanced melanoma, colorectal cancer, and non-small cell lung cancer. However, response rates towards ICB treatment varies widely across individuals and cancer types, leaving only a minority of patients benefiting from significant tumor regression and long-term protection from recurrence. While the exact mechanism of resistance is not fully understood, immunosuppression on multiple levels—genetic (e.g., impaired HLA class I and mutation of the PTEN gene), protein (e.g., VEGF, IL-10 and TGFβ), and cellular (e.g., inadequate antigen presentation and tumor infiltration of lymphocytes)—has been reported to be closely correlated to clinical outcome, suggesting the need for a more comprehensive approach in treatment.

To date, no FDA approval has been achieved for the widespread use of STING-agonist-based cancer treatments. This is primarily due to limitations in safety and efficacy, as prior studies have reported potential adverse effects such as dose-limited toxicity and a potent increase in serum interferon (IFN) levels. One challenge in terms of efficacy is the large human population with potentially defective STING due to genetic mutations. Studies have shown that the HAQ and H232 mutations of human STING, which account for ˜30% East Asian and ˜10% of European populations, are likely loss-of-function variants. As existing STING-targeting cancer immunotherapies rely on the intracellular delivery of STING agonists, the lack of fully functional endogenous STING could significantly abrogate signaling and treatment efficacy.

To this end, we previously developed an approach for cyclic guanosine monophosphate—adenosine monophosphate (cGAMP) delivery by adapting the cytosolic domain of STING protein (STINGΔTM) as a biomimetic carrier. The resulting STINGΔTM-cGAMP complex was shown in vitro to restore STING signaling in cell lines without STING protein or expressing only HAQ mutant STING, demonstrating its use as a fully functional protein^{52, 53}. In this work, we have engineered a novel fusion protein of ICB nanobody and STINGΔTM, which can then be complexed with cGAMP as an intratumorally-injected therapeutic. Through depletion studies, we observed that CD4⁺ T cells played a critical role in achieving antitumor immunity, to an extent that no significant differences were observed when compared to the untreated control. Mechanistic studies revealed a remodeling of the tumor microenvironment (TME) with proinflammatory cytokines, as well as the polarization of CD4⁺ T cells towards the T_{H}1 phenotype, followed by NK and CD8⁺ T cells—mediated cytotoxicity towards tumor cells. The ICB-STINGΔTM-cGAMP complex was found to effectively eradicate tumors in the MC38 colon carcinoma and the YUMM1.7 melanoma model, inhibit metastasis in the 4T1 breast cancer model, and prevent cancer recurrence in cured mice. Finally, when treating tumors on STING-knockout mice, the complex was able to achieve significant tumor regression via restored STING signaling with STINGΔTM. These results underscore the use of this combination in cancer immunotherapy, while simultaneously providing new insights regarding the potential benefits of STING-activated CD4⁺ T cells for clinical translation.

### Results

### Design and Characterization of the ICB-ΔTM-cGAMP Complexes

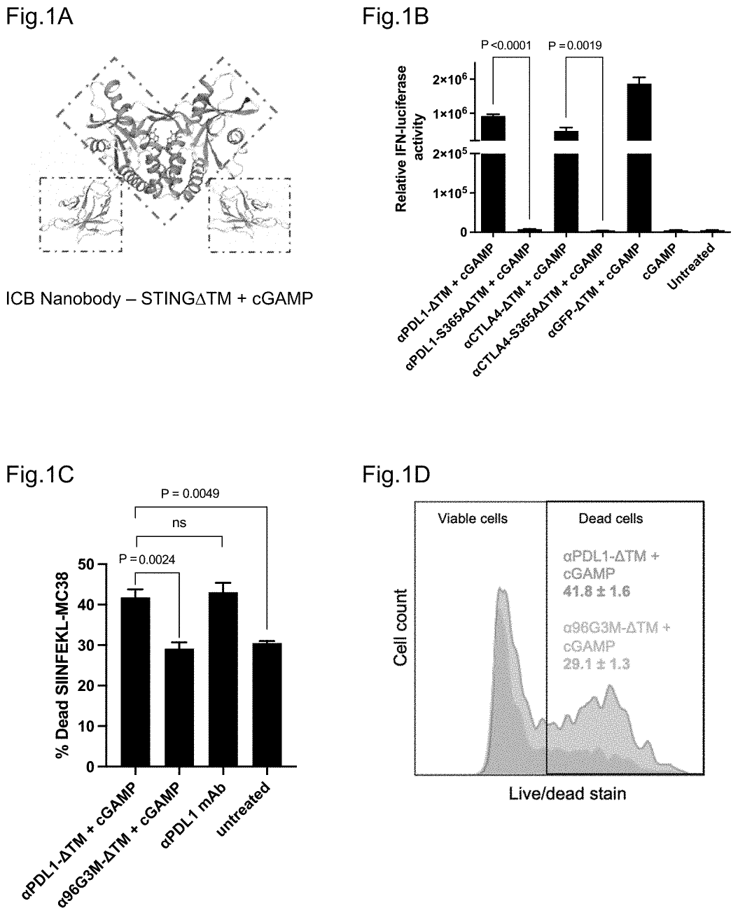

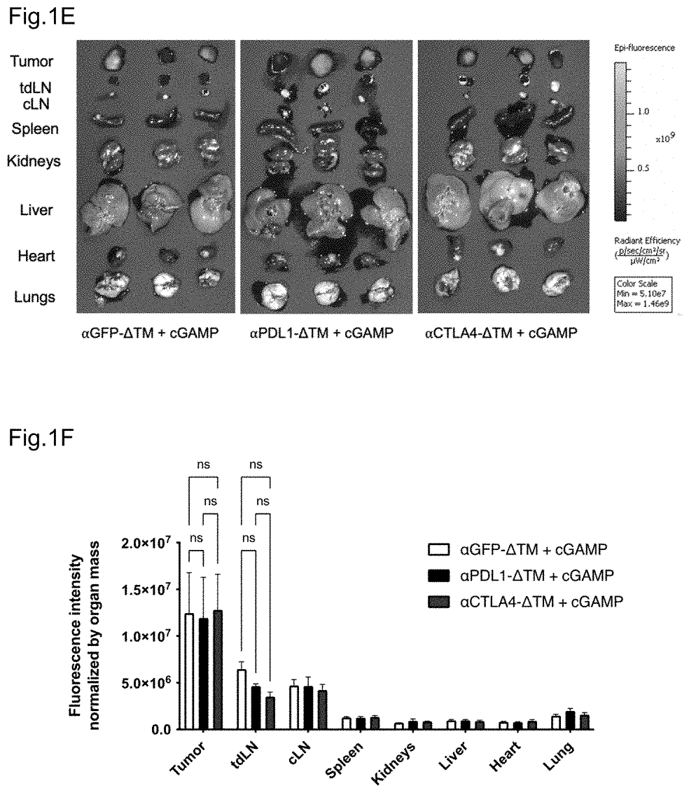

In engineering the ICB-STINGΔTM-cGAMP complex, we fused two types of ICB nanobodies to the STINGΔTM protein (ΔTM)—an anti-cytotoxic T-lymphocyte associated protein 4 (CTLA4) nanobody (αCTLA4-ΔTM), and an anti-programmed death ligand 1 (PDL1) nanobody (ΔPDL1-ΔTM) (FIG. 1A). The function of the complexes in restoring STING signaling was then evaluated in a human embryonic kidney (HEK293T) IFN-luciferase reporter cell line that is STING deficient (FIG. 1B), where they were observed to achieve high levels of interferon activity relative to agonist-only and controls of ΔTM (S365A mutant, which abrogates STING-mediated IFN signaling). Nanobody activity on the complex was likewise confirmed through an ex vivo cancer cell killing assay, in which SIINFEKL peptide-expressing MC38 cells were first stimulated with IFNγ to express PDL1 and then co-cultured with SIINFEKL-specific CD8⁺ T cells isolated from OT1 mice. Addition of the ICB-ΔTM-cGAMP complex resulted in a significant increase in the percentage of dead SIINFEKL-MC38 cells relative to a control nanobody-ΔTM targeting an irrelevant target 96G3M and untreated controls, as well as no significant difference when compared to the commercial anti-PDL1 monoclonal antibody (mAb), demonstrating full functionality of the immune checkpoint blockade (FIG. 1C and 1D). Subsequently, we recorded the biodistribution of the complexes through intratumoral delivery of cyanine 5 (Cy5)-labeled ICB-ΔTM-cGAMP (FIG. 1E and 1F). The complex remains almost exclusively within the tumor with some trafficking to the tumor draining lymph node (tdLN) 24 hours post-intratumoral injection, affirming the low probability of systemic toxicity through this route of administration.

ICB-ΔTM-cGAMP Eradicates Subcutaneous MC38 and YUMM1.7 Tumors with Immune Memory

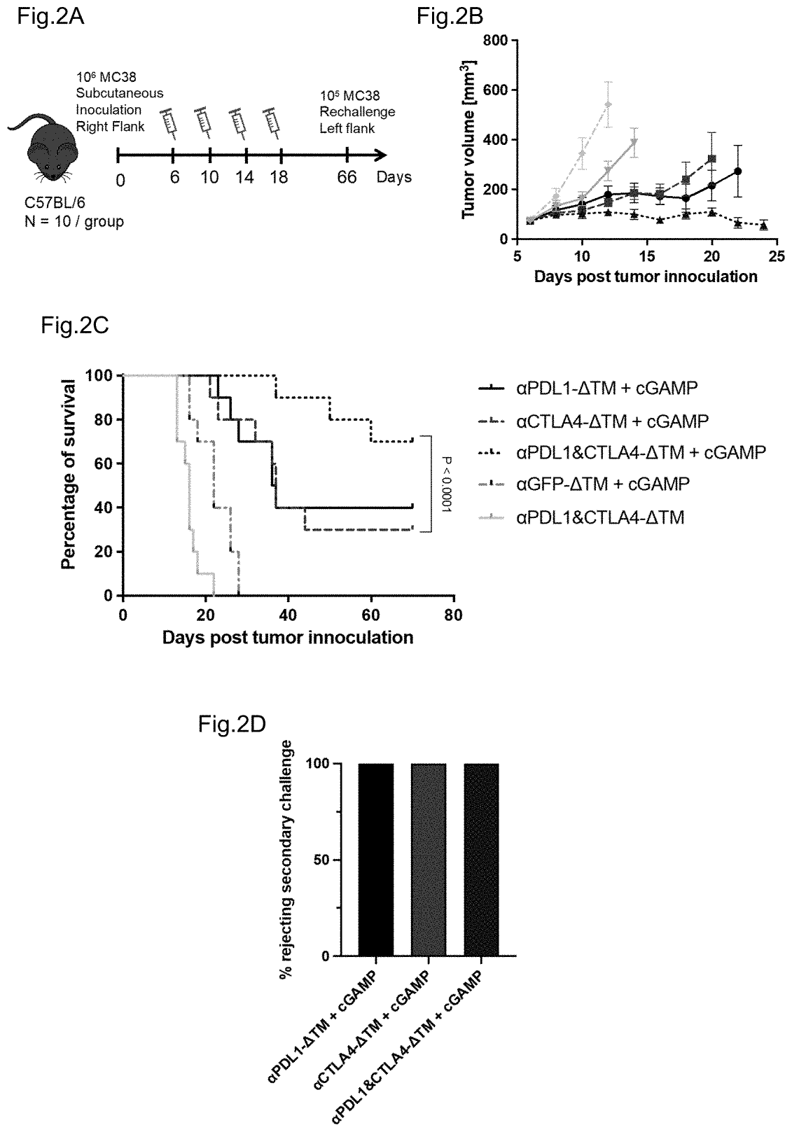

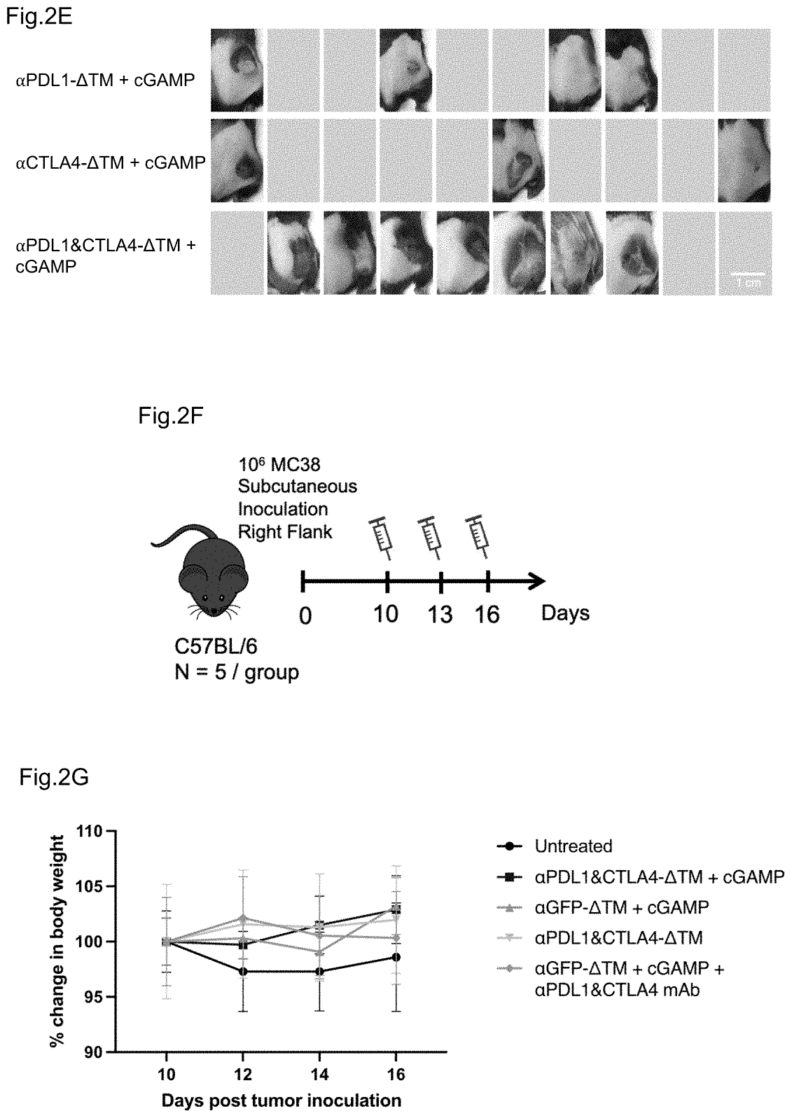

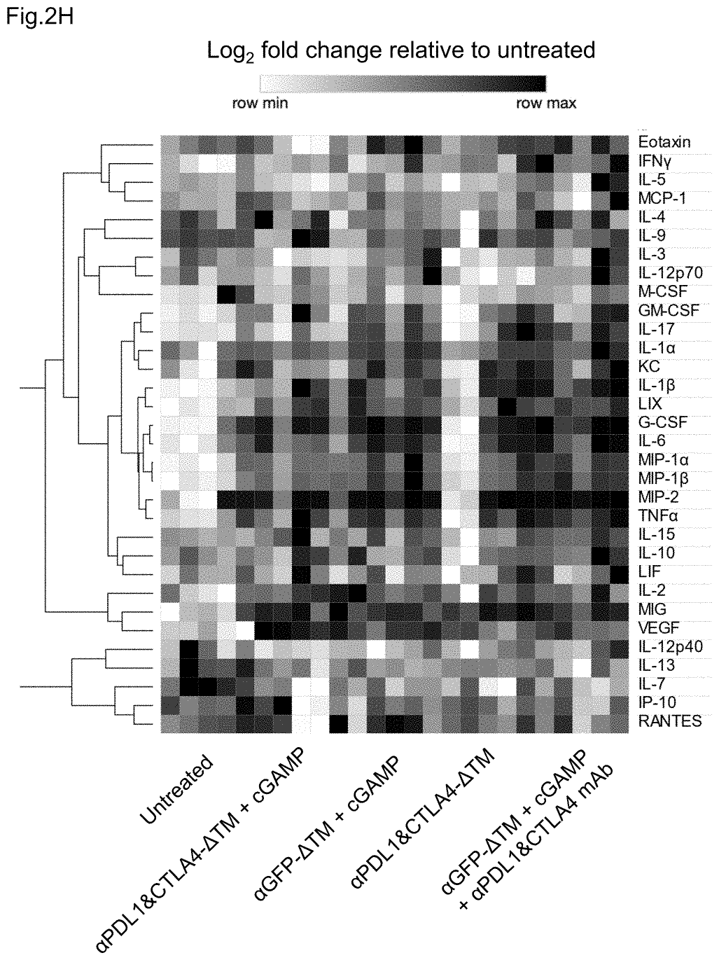

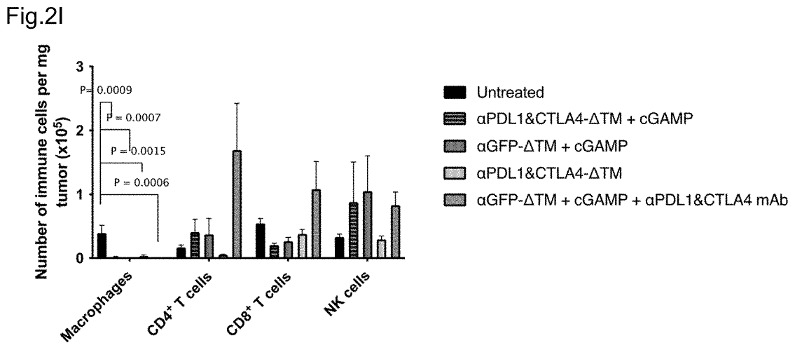

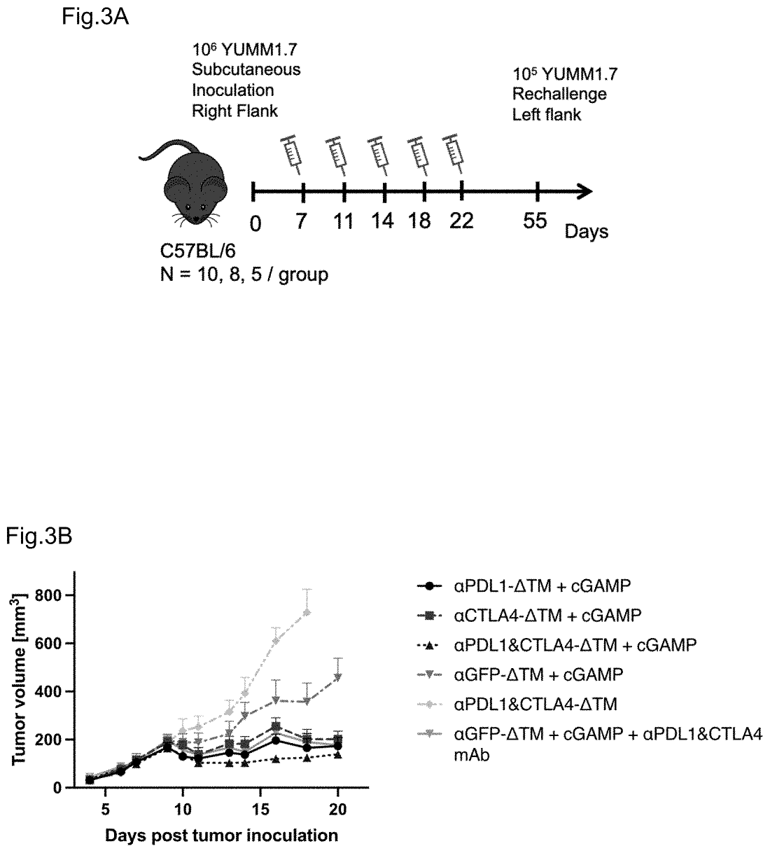

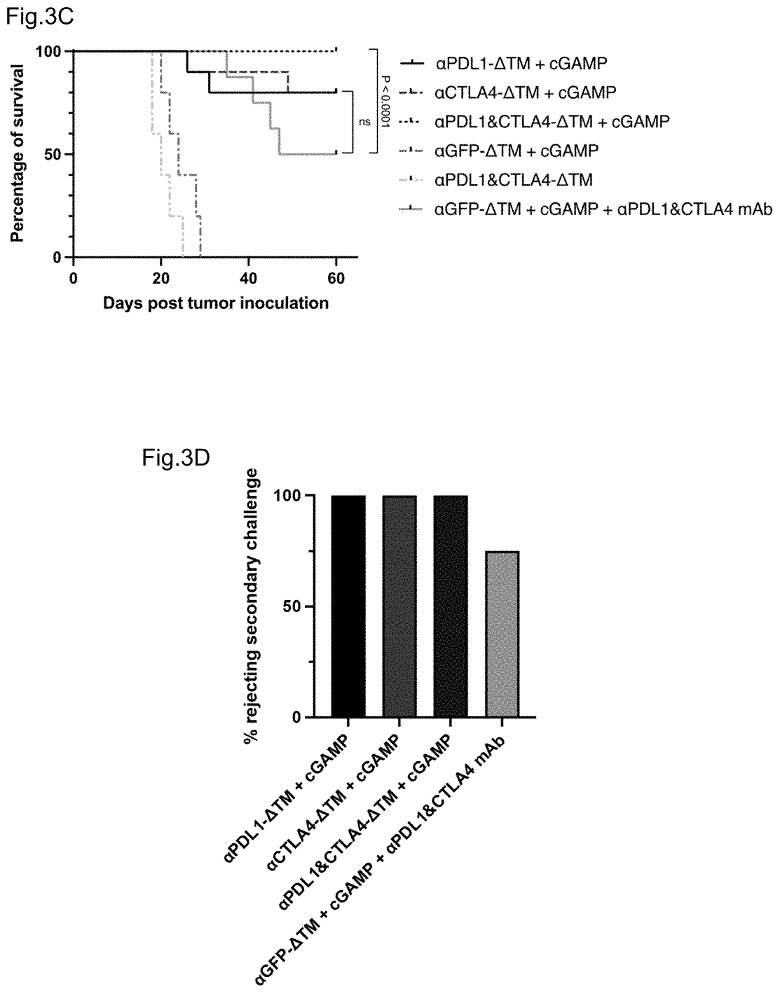

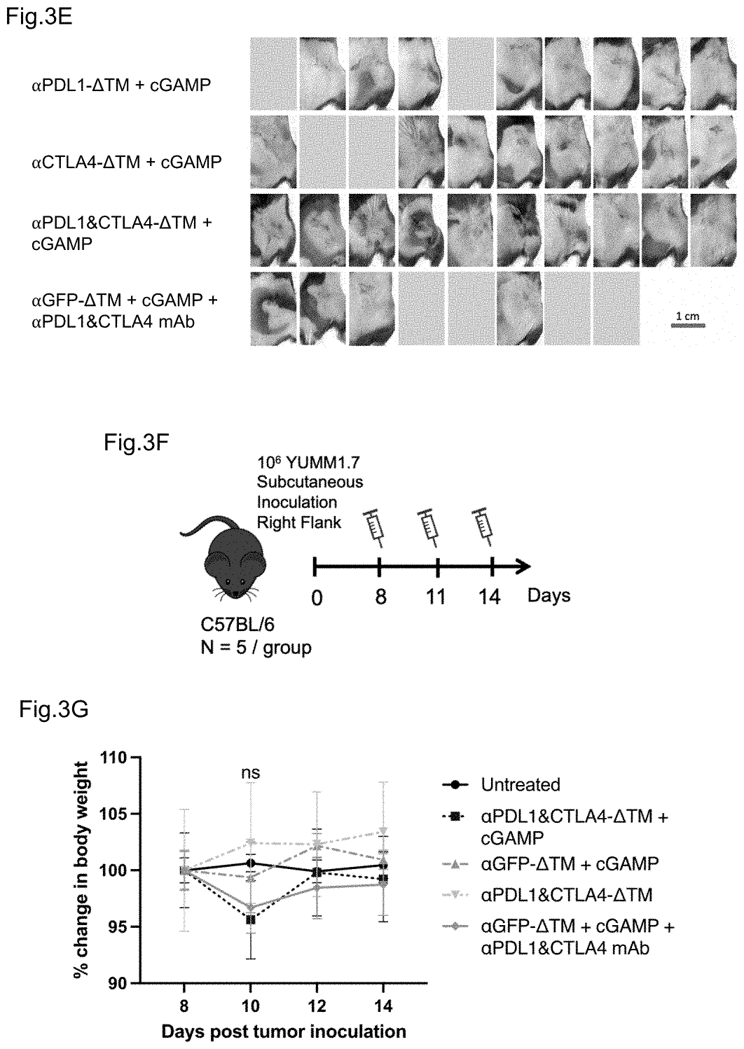

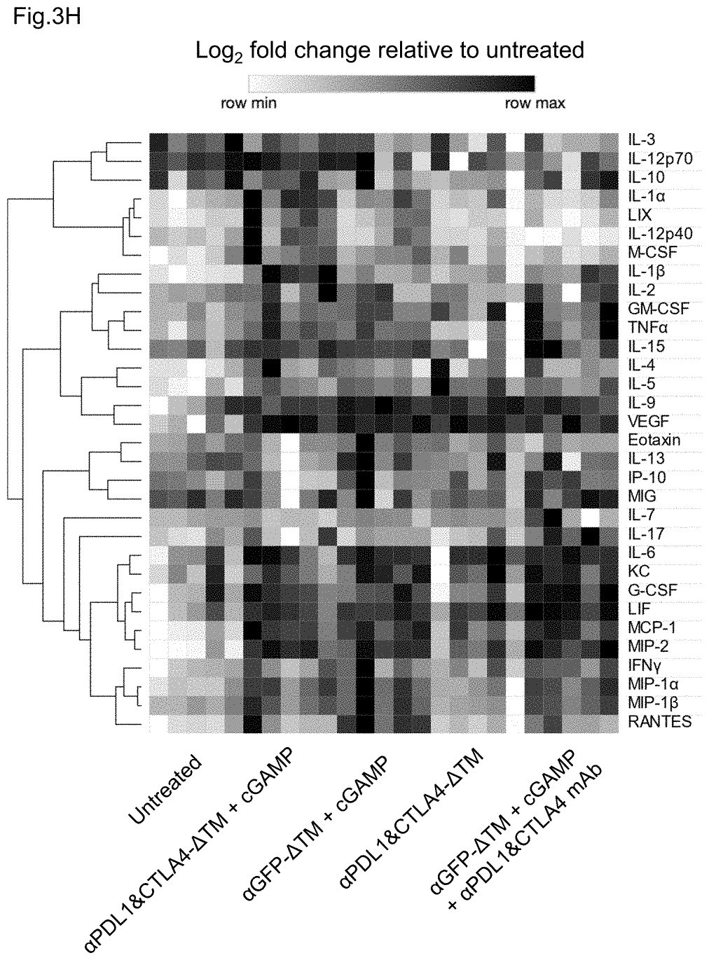

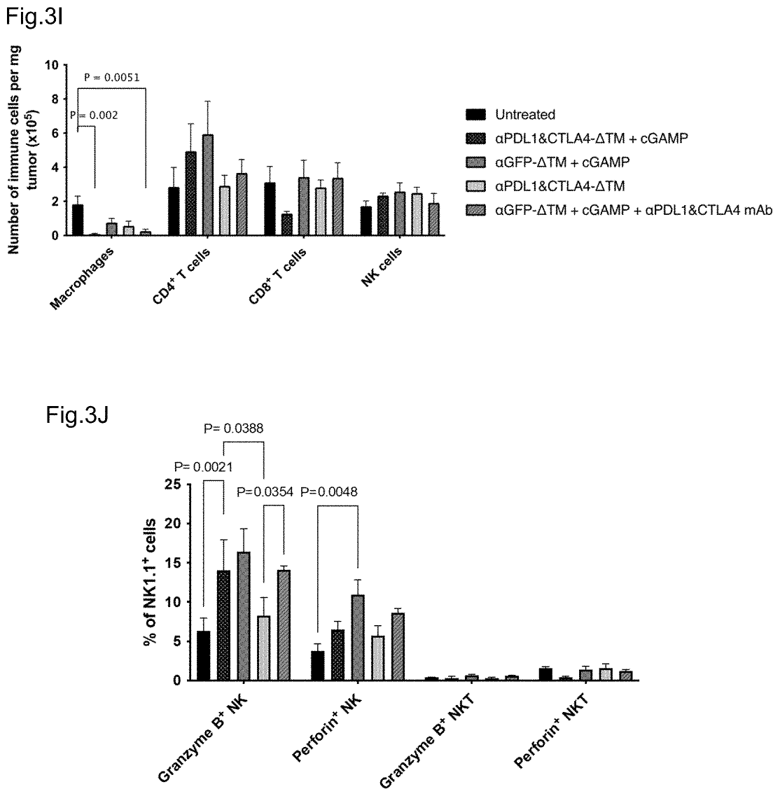

Next, we evaluated the therapeutic efficacy with two subcutaneous mouse tumor models, MC38 and YUMM1.7. We observed significant antitumor effect and sustained antitumor immune memory in both models following treatment with ICB-ΔTM-cGAMP (FIG. 2A and 3A). Five groups were included in the MC38 study: the individual ICB-ΔTM-cGAMP complexes (ΔPDL1-ΔTM-cGAMP and αCTLA4-ΔTM-cGAMP), an equal molar mixture of both complexes (ΔPDL1&CTLA4-ΔTM-cGAMP), a STING signaling-only control with nanobody targeting the green fluorescent protein (αGFP-ΔTM-cGAMP), and an ICB protein-only control without cGAMP (αPDL1&CTLA4-ΔTM). One additional group—STING with equal molar quantities of commercial ΔPDL1 and αCTLA4 mono antibodies (mAb) (αGFP-ΔTM-cGAMP+ΔPDL1&CTLA4 mAb)—was included in the YUMM1.7 experiment.

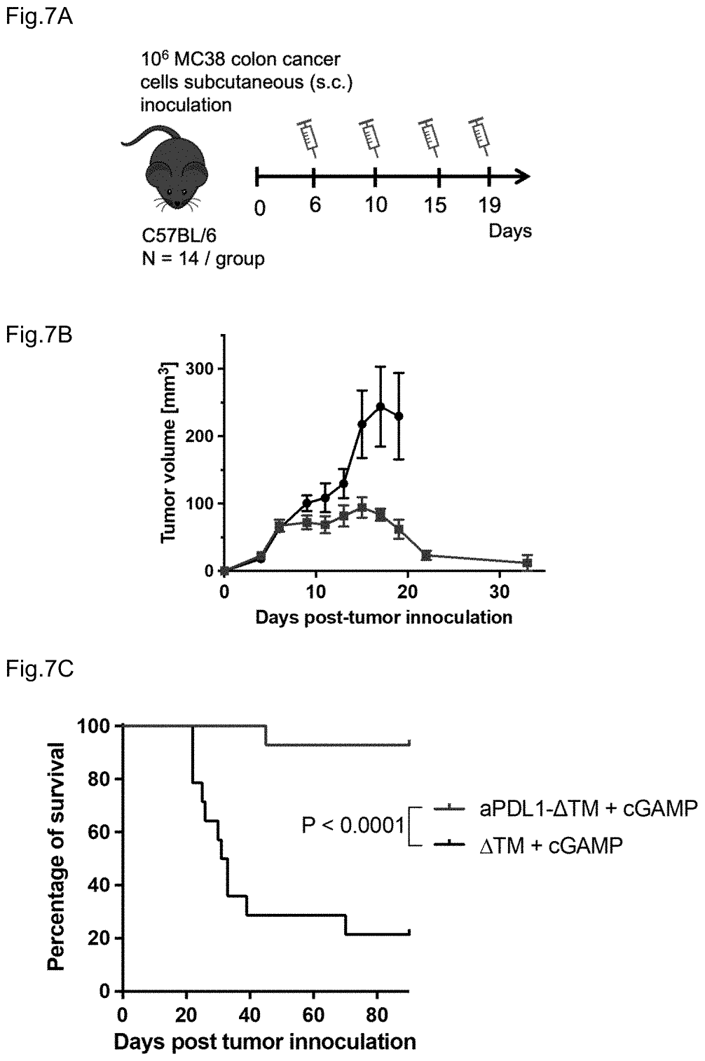

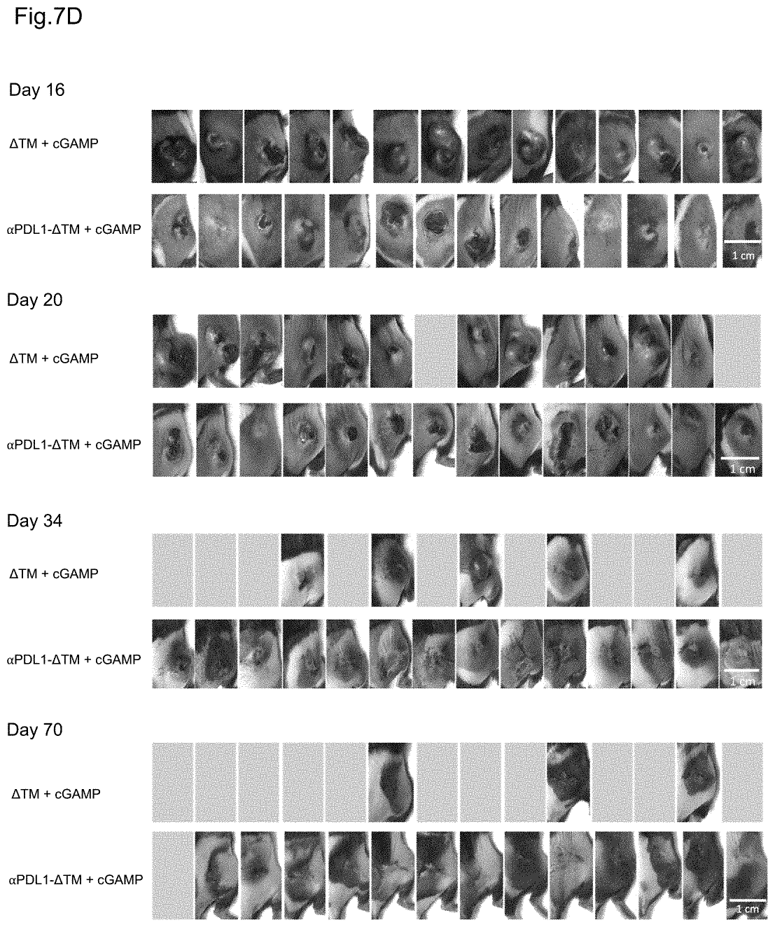

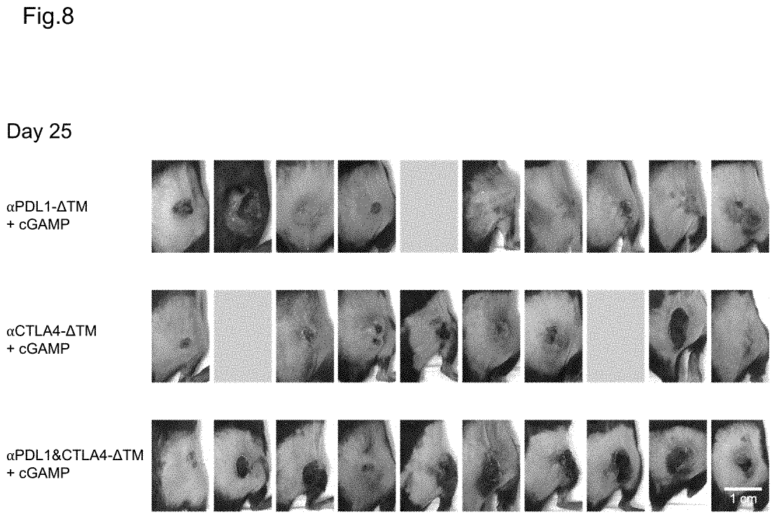

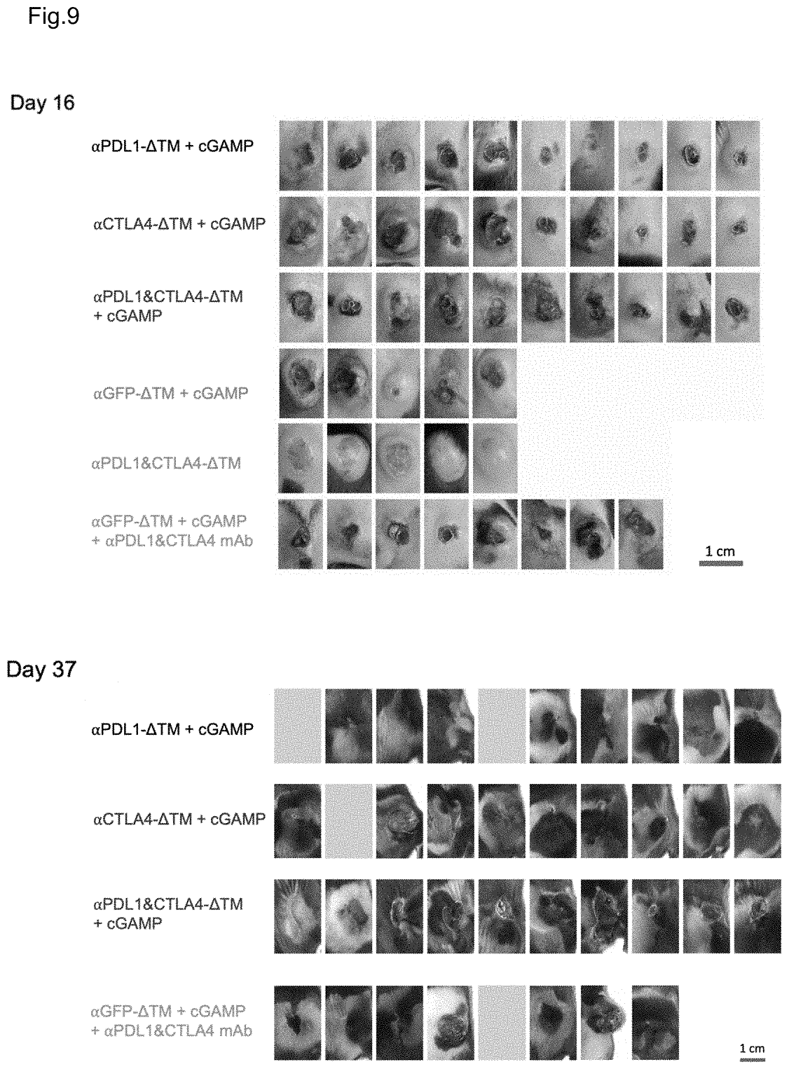

In both tumor models, all combinations of ICB-ΔTM-cGAMP were observed to significantly inhibit tumor growth and increase survival relative to STING-only (αGFP-ΔTM-cGAMP) and ICB-only controls (ΔPDL1&CTLA4-ΔTM). Tumors decreased in size and formed black scabs following treatment, which sloughed off and healed without further intervention (FIG. 7). We also noted that therapeutic benefits from enhanced STING signaling typically occurred earlier than those from ICB; for instance, MC38 tumors treated with cGAMP exhibited tumor growth inhibition around Day 10 in comparison to the protein-only group, while ICB-ΔTM-cGAMP formulations only began to outperform STING-only treatments at Day 14. This effect was corroborated in the YUMM1.7 melanoma model, where significant differences between the ICB-only group and ICB-ΔTM-cGAMP treatments could be observed at Day 12, whereas the benefit of checkpoint blockade was only evident at around Day 15. In particular, the dual blockade group αPDL1&CTLA4-ΔTM-cGAMP resulted in the most prominent survival benefit (7/10 and 10/10 for MC38 and YUMM1.7, respectively), more so than that of single blockade groups ΔPDL1-ΔTM-cGAMP and αCTLA4-ΔTM-cGAMP (FIG. 2B, 2C, 3B 3C, 8, and 9). All survivors from both studies treated with ICB-ΔTM-cGAMP combinations also exhibited 100% tumor rejection upon rechallenge on the opposite hind flank, while αGFP-ΔTM-cGAMP+ΔPDL1&CTLA4 mAb resulted in 75% tumor rejection (FIG. 2D and 3D). All survivors remained tumor-free until the end of the six-month observation period, with representative images of healed skin provided in FIG. 2E and 3E.

To characterize the TME, we performed separate treatment experiments for both MC38 and YUMM1.7 models, resecting the tumors from mice after the 3^{rd} dose for analysis (FIG. 2F and 3F). Body weight was also monitored over the course of the treatments. ICB-ΔTM-cGAMP therapy led to a maximum weight loss of ˜5%, though this difference was insignificant compared to untreated groups (FIG. 2G and 3G). Flow cytometry analysis of the excised tumors (FIGS. 2I and 3I) revealed that ICB-ΔTM-cGAMP therapy led to a lower average percentage of tumor associated macrophages (TAMs), which could be beneficial to tumor treatment as they are generally associated with immunosuppression and poor prognosis⁵⁴. We also observed upregulated pro-inflammatory cytokines in the TME: IL-2, MIP-2, IL-15, G-CSF for MC38 and IL-2, IL-12, IL-15, G-CSF, TNFα for YUMM1.7 (FIG. 2H and 3H). Finally, we characterized the NK and NKT cells in the YUMM1.7 model and found that ICB-ΔTM-cGAMP therapy could effectively activate the cytotoxicity of NK cells, as demonstrated by the upregulated levels of granzyme B and perforin in NK cells (FIG. 3J).

ICB-ΔTM-cGAMP Engages CD4⁺ T, NK and CD8⁺ T Cells in a Multi-Pronged Immune Response

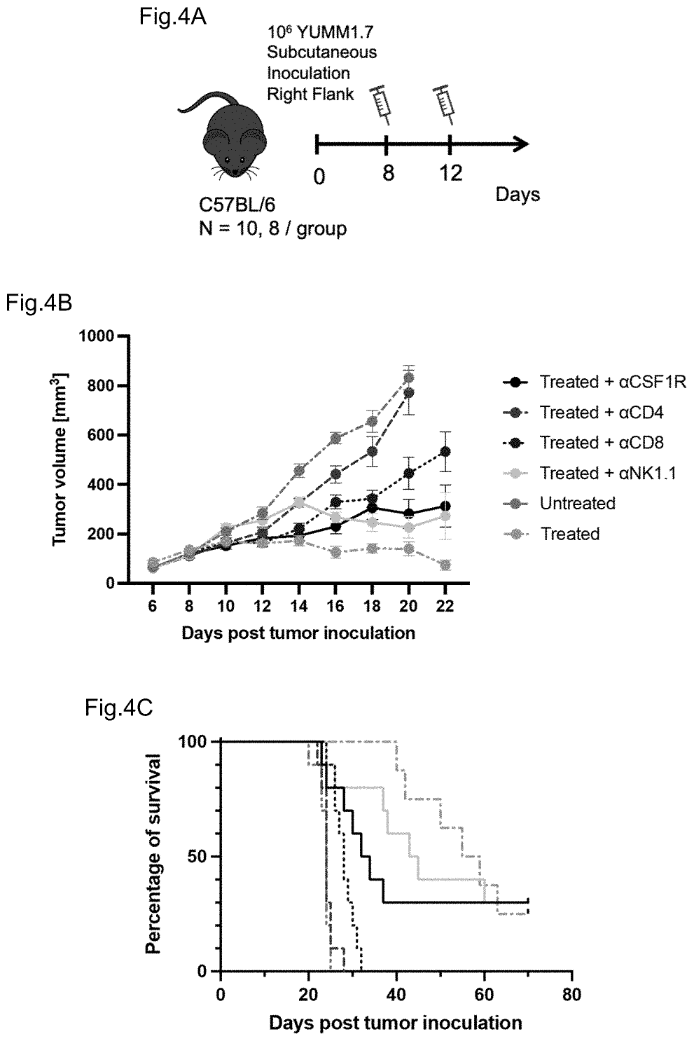

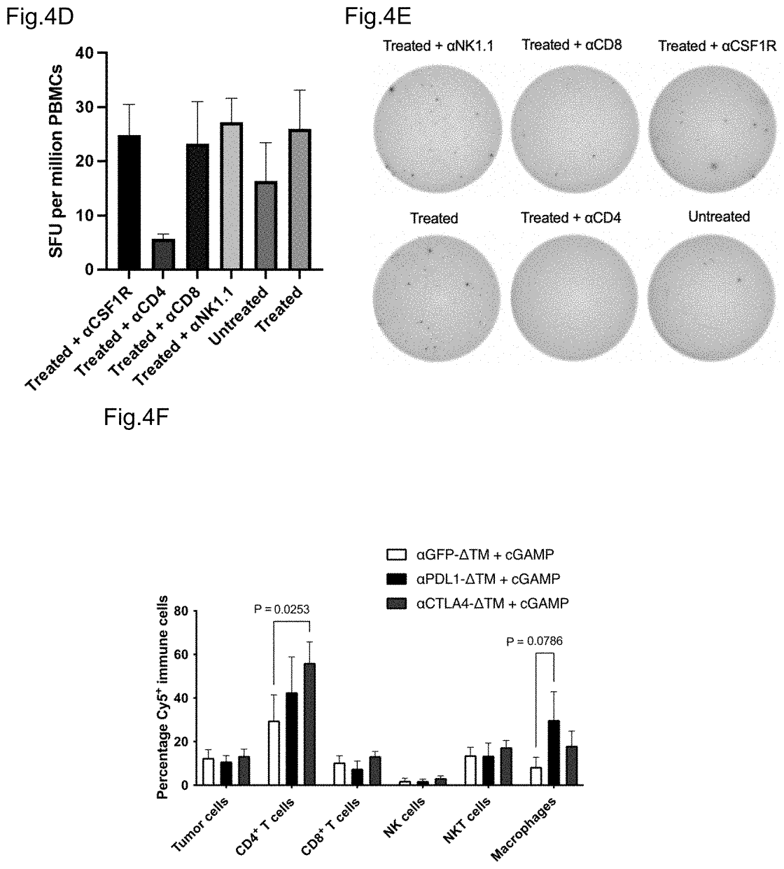

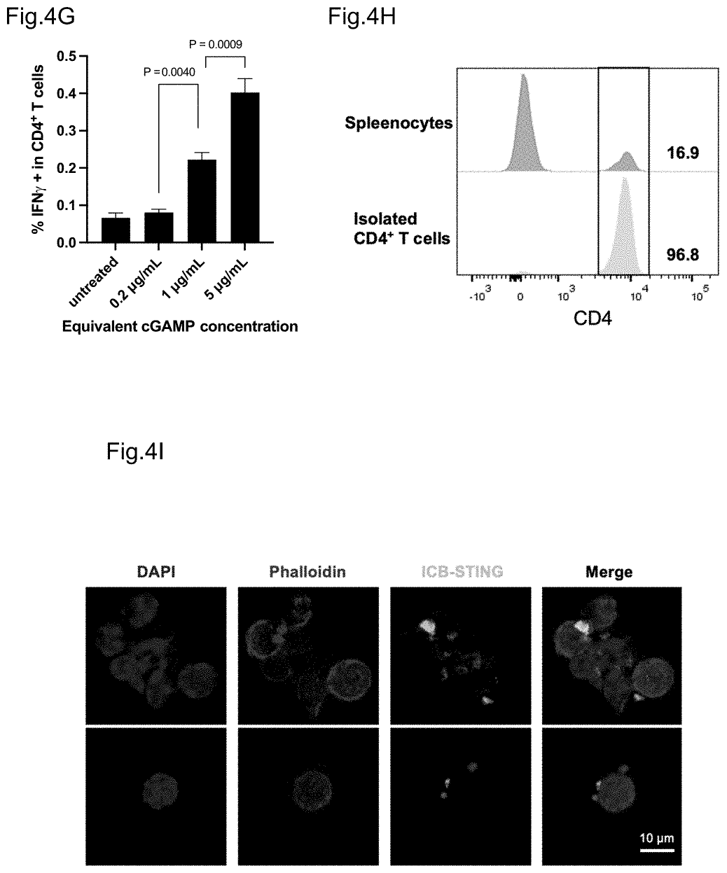

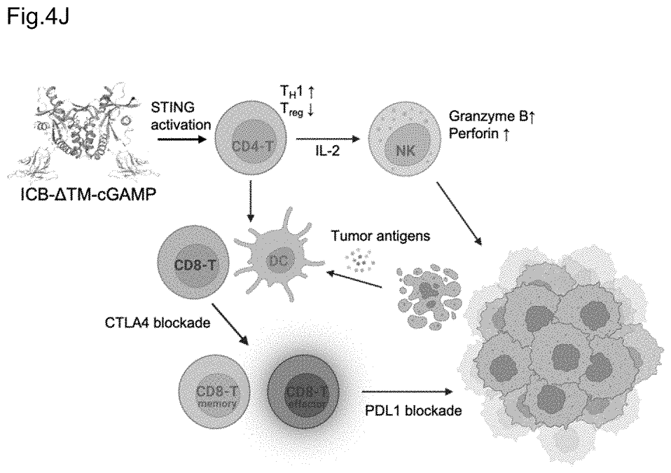

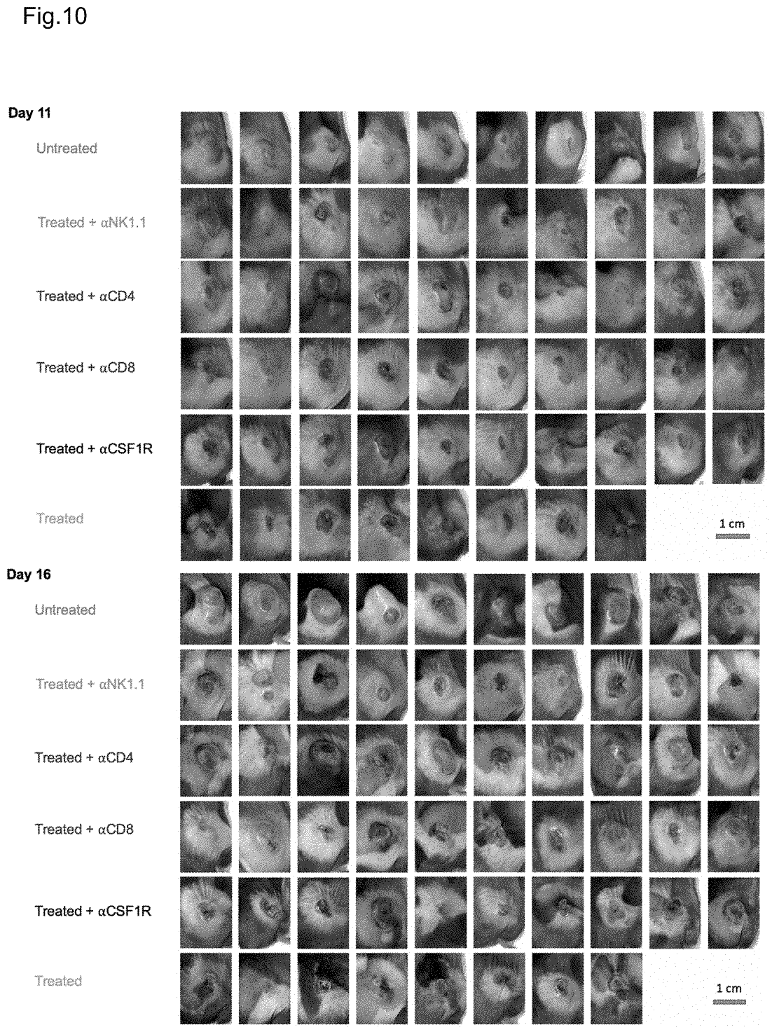

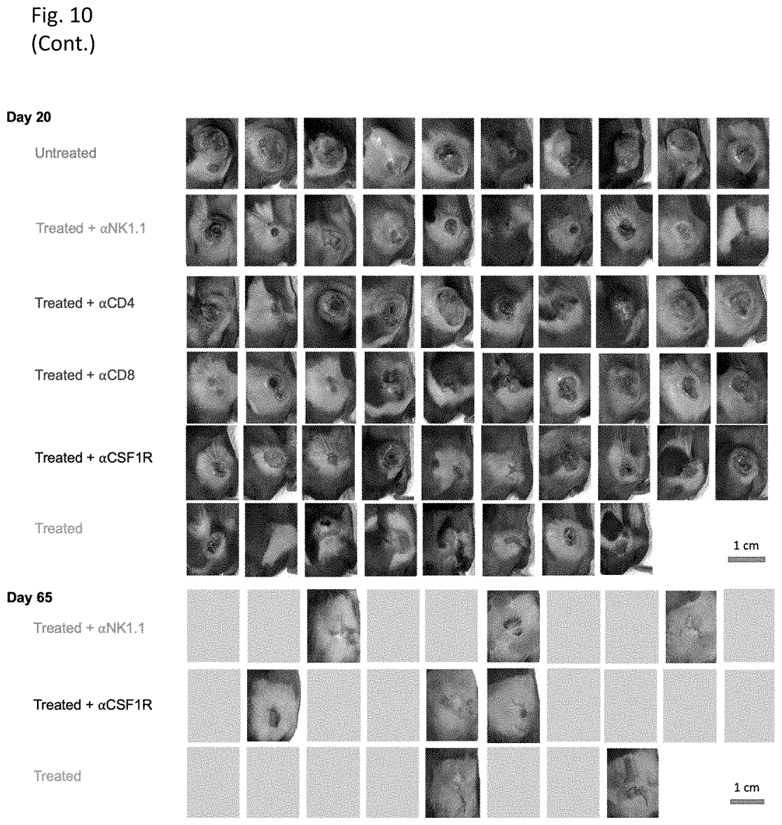

The therapeutic mechanism of ICB-ΔTM-cGAMP was observed to rely significantly on CD4⁺ T, NK, and CD8⁺ T cell-mediated pathways. Analysis of MC38 and YUMM1.7 tumors first revealed a significantly larger population of NK cells in the αPDL1&CTLA4-ΔTM-cGAMP treatment groups when compared to STING-only or ICB-only trials. These results were corroborated by an immune cell depletion study with YUMM1.7 model (FIG. 4A), where the depletion of NK and NKT cells with αNK1.1 antibodies initially resulted in a steep increase in tumor growth. The trend persisted up to two weeks post-tumor inoculation before plateauing, just as tumors on CD8⁺ T cell-depleted mice began to drastically increase in size (FIG. 4B, FIG. 10).

Despite the initial anti-tumoral effect of NK cells, the overall survival of the NK-depleted group indicated that NK cells were not essential for eradication of the tumor. Both NK and macrophage-depleted groups failed to exhibit any significant differences in overall survival compared to the no-depletion control, with all groups achieving a survival rate of 25%-30% (FIG. 4C). In contrast, CD4⁺ and CD8⁺ T cells appeared to be indispensable for antitumor immunity, as their depletions fully abolished therapeutic efficacy from the treatment. Some tumor inhibition was observed for the CD8-depleted group during Day 10-15 of treatment, coinciding with the period where NK-depleted mice experienced the steepest increase in tumor growth, suggesting that NK cells were responsible for the initial suppression of tumor growth in CD8-depleted mice. However, this effect was transient: after Day 15, we observed a drastic increase in tumor volume in the CD8-depleted group due to lack of antitumoral immunity, coinciding with decreased tumor growth in NK-depleted mice. Altogether, these results provide evidence for the temporal progression of first NK and then CD8⁺ T cell-mediated antitumor immunity in response to treatment.

Most notably, the depletion of CD4⁺ T cells proved to be critical to the therapeutic effect of ICB-ΔTM-cGAMP at all stages of the study, resulting in tumor progression similar to that of untreated controls. In comparison with NK- and CD8-depleted groups, CD4-depleted group exhibited no therapeutic benefit from either NK or CD8⁺ T cells throughout the study. To investigate this phenomenon, we sought to quantify the amount of tumor antigen-specific immune cells from peripheral blood mononuclear cells (PBMCs) via IFNγ ELISPOT and observed significantly compromised adaptive immunity in CD4-depleted mice, worse even than that of the untreated group (FIG. 4D and 4E).

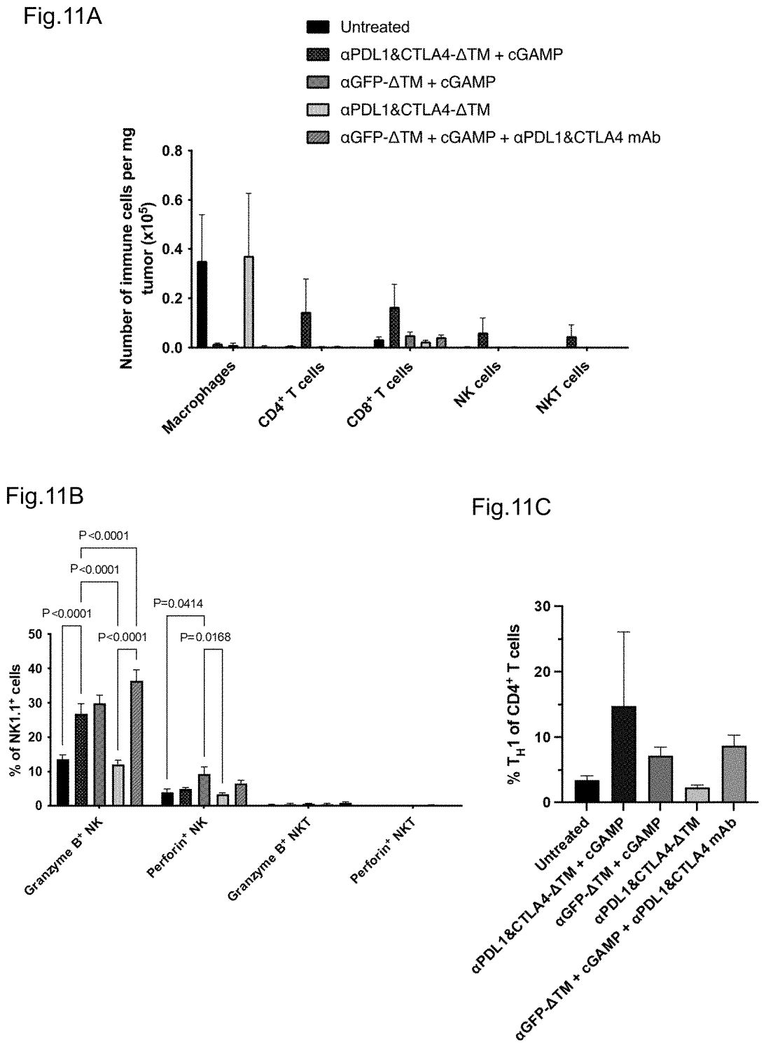

The aforementioned results led us to speculate that the engagement of CD4⁺ T cells with ICB-ΔTM-cGAMP occurs at an early timepoint. To verify this hypothesis, we sought to characterize tumor immune cells 24 hours post-intratumoral injection of Cy5-labeled ICB-ΔTM-cGAMP. Flow cytometry analysis of the excised tumors demonstrated the cellular targeting of ICB nanobodies—αCTLA4 towards CD4⁺ T cells and ΔPDL1 towards macrophages. Overall, the highest percentage Cy5⁺ cells were the CD4⁺ T cells (FIG. 4F), indicating close interaction with ICB-ΔTM-cGAMP. We also observed CD4⁺ T cell T_{H}1 polarization as well as NK-mediated cytotoxicity in treatment groups with cGAMP, demonstrating the early anti-tumoral immunity from STING signaling (FIG. 11). To further explore this interaction, we treated the splenocytes of C57BL/6 mice with ΔPDL1&CTLA4-ΔTM-cGAMP ex vivo and analyzed the T_{H}1 polarization of CD4⁺ T cells 24 hours post-treatment. We observed that higher concentrations of ΔPDL1&CTLA4-ΔTM-cGAMP led to higher polarization towards the T_{H}1 phenotype (FIG. 4G) and sought next to decouple this effect from other immune cells by repeating the experiment on CD4⁺ T cells isolated from splenocytes (FIG. 4H). This was also found to result in enhanced T_{H}1 polarization, with confocal micrographs corroborating increased interaction (FIG. 4I).

At this juncture, a therapeutic mechanism for the intratumoral delivery of ICB-ΔTM-cGAMP may be proposed. During the first 2 weeks, CD4⁺ T and NK cells are major contributors to tumor regression. The injected ICB-ΔTM-cGAMP primarily interacts with CD4⁺ T cells, giving rise to T_{H}1 polarization, a subtype known for its supporting role in inducing cellular immunity, including activating and sustaining NK cells via IL-2 secretion⁵⁵. On the other hand, while NK cells did not directly interact with ICB-ΔTM-cGAMP, our intracellular staining of granzyme B and perforin suggested a robust activation of NK cytotoxicity. We therefore hypothesize that CD4⁺ T cells play a role in stimulating NK-mediated anti-tumor immunity, though the specific mechanism remains to be uncovered. Concurrently, CD4⁺ T cells aid in antigen presentation and in priming CD8⁺ T cells, which leads to CD8⁺ T cell-mediated antitumor immunity at 2-3 weeks post-injection and CD8⁺ memory T cell-based protection against tumor recurrence (observed in YUMM1.7 and MC38 tumor models). The process has been illustrated in FIG. 4J.

### ICB-ΔTM-cGAMP Inhibits Metastasis in 4T1 Model

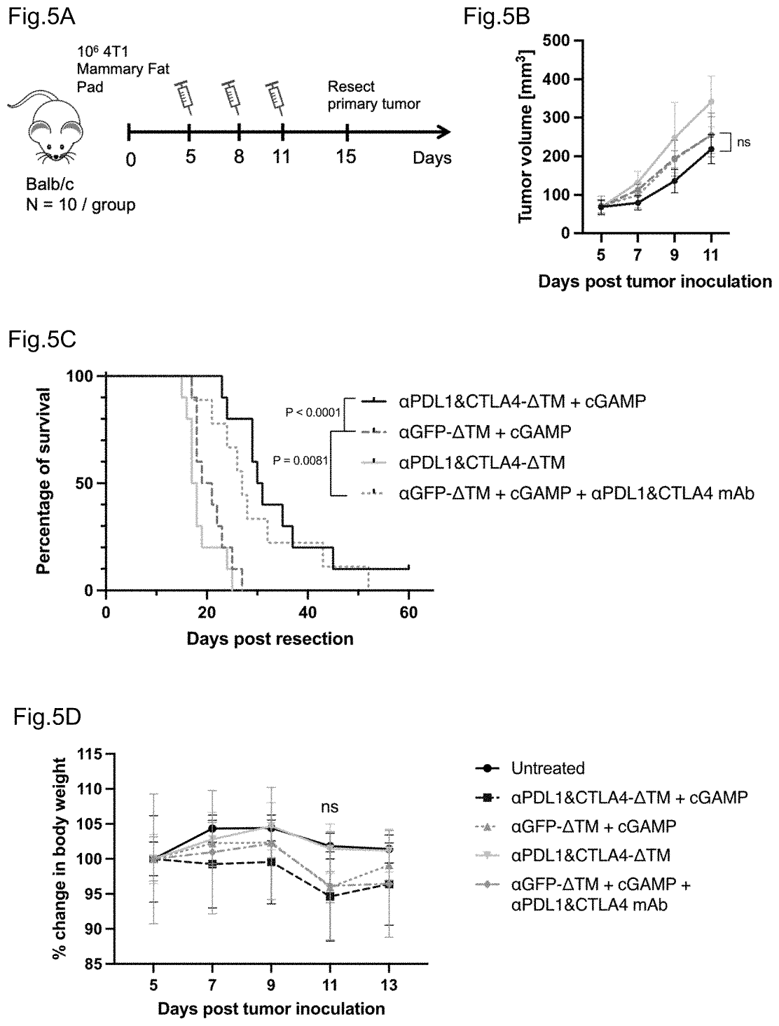

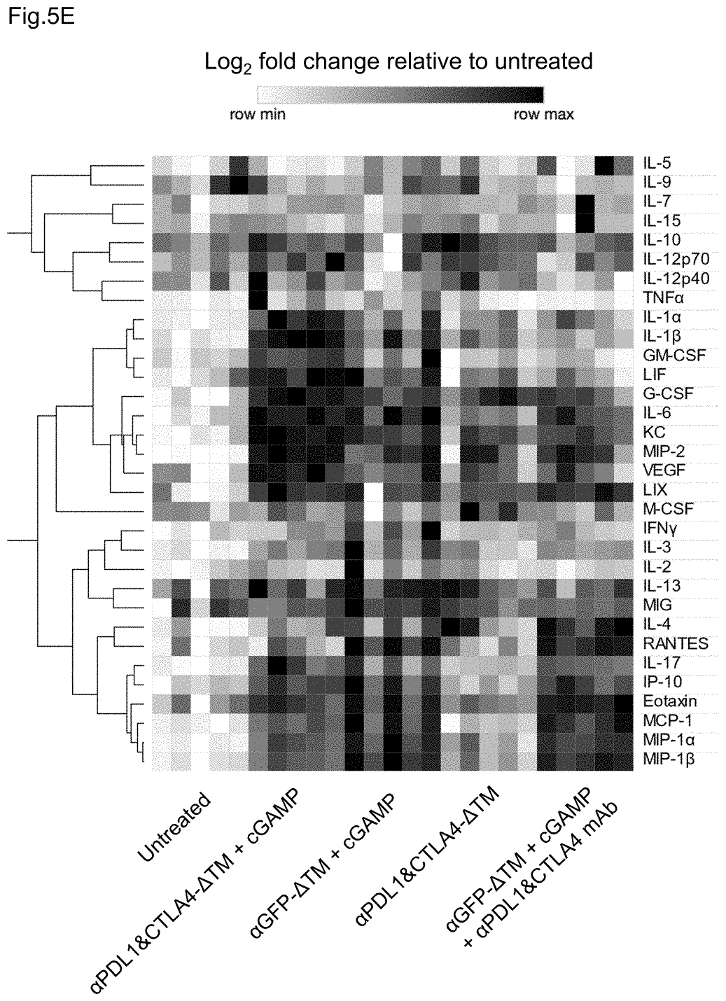

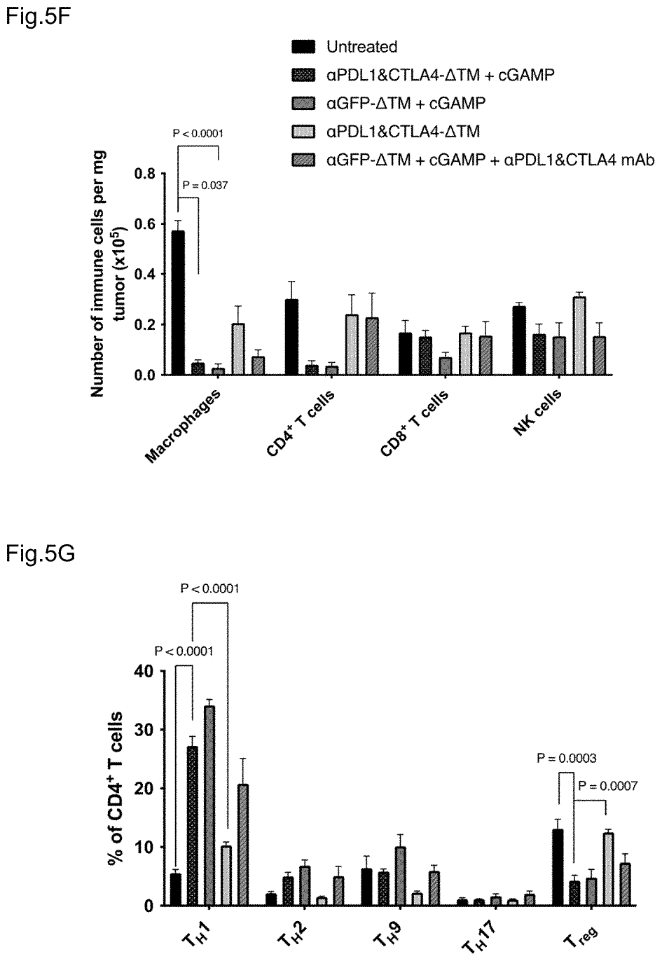

To assess the systemic immune protection afforded by the intratumoral delivery of ICB-ΔTM-cGAMP, we next treated the 4T1 metastatic breast cancer model (FIG. 5A). When no significant decrease in tumor volume was observed following three intratumoral doses of ΔPDL1&CTLA4-ΔTM-cGAMP, the primary tumor was surgically resected. The rate of lung metastasis was then monitored indirectly via post-op survival, where ΔPDL1&CTLA4-ΔTM-cGAMP-treated mice survived for significantly longer than those dosed with STING-only or ICB-only controls (FIG. 5B and 5C). Analysis of resected tumors revealed a significant decrease in TAMs relative to untreated controls in the ΔPDL1&CTLA4-ΔTM-cGAMP treatment group. The resected tumors also exhibited significant T_{H}1 polarization of CD4⁺ T cells and suppression of Treg (FIG. 5G), while cytokine analysis of the tumors suggested increased inflammation in the TME (as evidenced by higher levels of G-CSF, GM-CSF, IL-1α and IL-12). These results underscore the critical role of CD4⁺ T cells in the treatment, as well as its benefit in both metastatic and non-metastatic tumor models.

### ICB-ΔTM-cGAMP Restores STING Signaling in STING Deficient Host

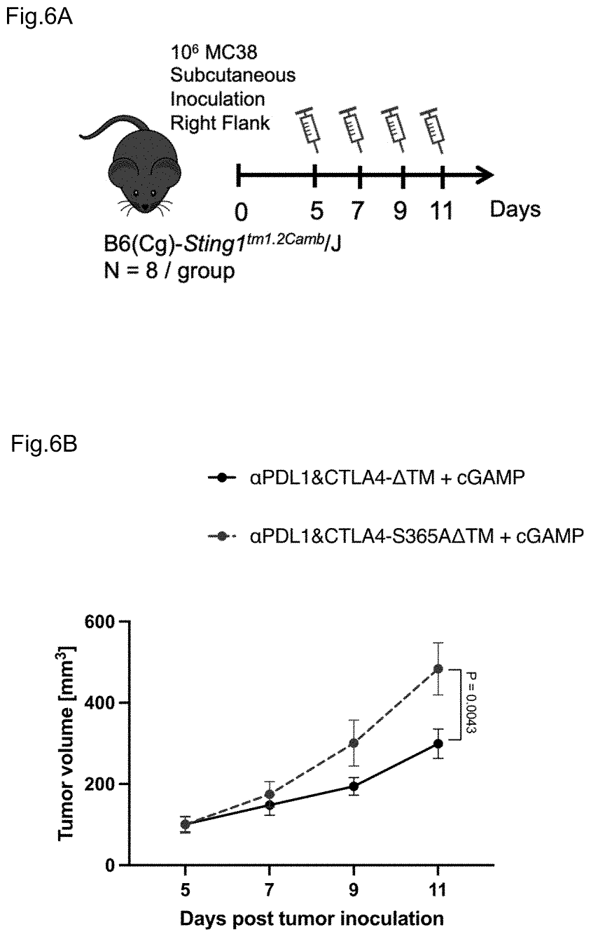

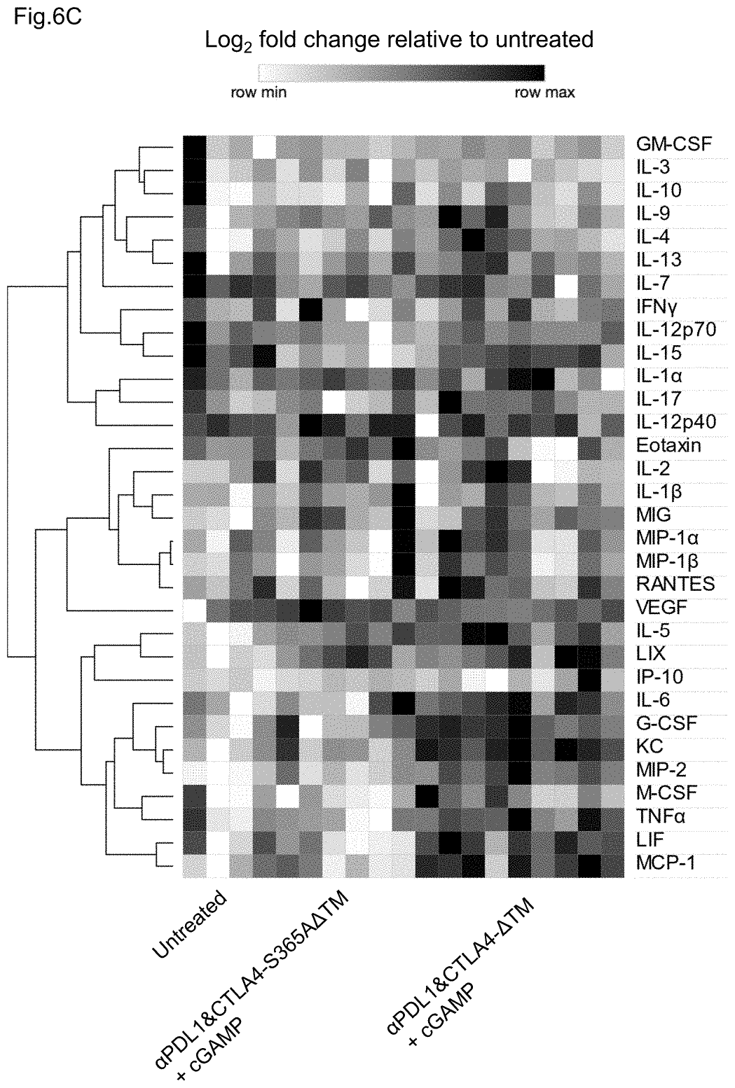

Finally, we explored the possibility of restoring STING signaling with the functional STINGΔTM carrier by treating MC38 tumors on STING KO mice with ΔPDL1&CTLA4-ΔTM-cGAMP. To decouple immune responses against this protein complex not induced via the STING pathway, we included ΔPDL1&CTLA4-S365AΔTM-cGAMP as a control (FIG. 6A) and increased the dosing frequency to account for lower therapeutic efficacy relative to immunocompetent mice. After four doses, we were able to observe a significant reduction in tumor volume relative to the S365A control, indicating that this result is due entirely to the STING-activating functionality of the STINGΔTM carrier (FIG. 6B). This was further confirmed through analysis of the cytokine profile in resected tumors, where we observed upregulated IL-12, IL-15, IL-13, G-CSF, TNFα, KC, etc. This was consistent with the cytokine profiles of the MC38, YUMM1.7 and 4T1 models (FIG. 6C), and likewise with the general profiles of STING activation in literature³³.

### Discussion

Potent immunostimulation as well as effective approaches to reverse immunosuppression and capture tumor cells that evade immunosurveillance remains a challenge in clinical cancer immunotherapy

The cancer immunity cycle has traditionally been depicted as occurring between dendritic cells, which prime CD8⁺ T cells with tumor antigens, resulting in the elimination of tumor cells and the generation of antigens in self-sustaining loop. In recent years, NK cells have entered the picture, collaborating alongside CD8⁺ T cells. Here, we engineered a new strategy to effectively boost the cancer immunity cycle, elucidating the critical role of CD4⁺ T cells as an initiator for NK cell tumor elimination⁷⁰ and an aide to CD8⁺ T cell priming and memory⁷¹ in STING-mediated antitumor immunity. This is a highly salient observation, as the role of CD4⁺ T cells in cancer immunotherapy has been reported to be bidirectional. Despite evidence that CD4⁺ T cells are fundamentally important in supporting anti-tumoral immunity², many studies over the past two decades have shown that CD4⁺ T cell depletion results in improved therapeutic outcomes, including a wide range of therapies such as adoptive T cell transfer, inflammatory cytokines, and ICB antibody treatments⁷. There has even been a human clinical trial intravenously injecting anti-CD4 depletion antibodies to treat solid tumors, where tumor shrinkage was observed in 5/11 patients over three months of observation⁷⁸. In these therapies, the presence of CD4⁺ T cells did not positively contribute to antitumor immunity, which was attributed to the immunosuppressive effects of T_{reg} and T_{H}2 outweighing that of T_{H}1.

While the positive role of CD4⁺ T cells has not been extensively investigated in many immunotherapies, recent studies have suggested that significant potential benefit could be exploited from CD4⁺ T cells if properly activatedln our study, we observed depletion of CD4⁺ T cells. Additionally, we found that CD4⁺ T cells could directly interact with our STING signaling complex ex vivo (FIG. 4G-4I). This resulted in effective suppression of the undesirable T_{reg} within the TME while achieving ˜30% T_{H}1 polarization of CD4⁺ T cells in vivo (FIG. 5G). The overall role of CD4⁺ T cells in our treatment was demonstrated to be very beneficial. Furthermore, this approach results in the suppression of TAMs, as well as the generation of multiple pro-inflammatory cytokines. Altogether, the therapy disclosed herein initiates a strong, collaborative response among various types of immune cells, with positive feedback loops to reverse immunosuppression and sustain antitumoral immunity.

Following initiation of the innate immune system via STING-mediated CD4 T_{H}1-polarization and NK-mediated cytotoxicity, the ICB-ΔTM-cGAMP complex then acts upon the adaptive immune system through ICB. We sought to leverage the synergy of PDL1 and CTLA4 dual blockade and succeeded in eradicating both immunologically “cold” (YUMM 1.7⁸³) and “hot” (MC38⁸⁴) tumors, gaining long-term protection against recurrence through memory T cells. When administrated as a neoadjuvant for the metastatic 4T1 model—a model recalcitrant to immunotherapy—our approach likewise resulted in prolonged post-op survival. These results demonstrate this system's utility as a modular platform for incorporation of other ICB or tumor-targeting nanobodies, such as anti-TIGIT and anti-NGK2A for NK cells, anti-PSGL-1 for CD4⁺ T cells, anti-EIIIB for tumor extracellular matrices⁸⁵, allowing additional flexibility in treating a wide range of cancers.

One remaining challenge to clinical translation lies in host STING deficiency and the limited options to restore STING signaling in affected individuals. Our present system provides a tangible solution by eliciting significant antitumoral immunity via STING signaling in STING KO mice, both in terms of tumor burden and in the production of pro-inflammatory cytokines within the TME. The aforementioned findings emphasize this platform's immense utility in CD4⁺ T cell activation and overcoming host STING deficiency, offering new solutions to improving the clinical outcomes of ICB treatments.

### Materials and Methods

### Protein Purification

The expression plasmid was cloned based on our previous pSH200_STINGΔTM plasmid, where STINGΔTM stands for the segment of amino acids 138-378 of mouse STING⁵². DNA sequences encoding for nanobodies of anti-PDL1 (PDB ID: 5DXW_A), anti-CTLA4 (PDB ID: 5E03_A), anti-GFP (PDB ID: 3OGO_E), anti-96G3m (PDB ID: 6X07_B) were synthesized by gBlock (IDT) and inserted at the N-terminus of the sequence encoding for STINGΔTM protein. Mutants such as S365A and R237A/Y239A of the STINGΔTM protein were created via site-specific mutagenesis. Histidine₆-tagged nanobody-STINGΔTM proteins were expressed in Rosetta DE3 Escherichia coli (Rosetta E. coli, Millipore Sigma, Cat#: 70954). The purification method is slightly modified based on our previous published protocol⁸⁶. Briefly, Rosetta E. coli transformed with the protein expression plasmid were cultured in 1 L volume of lysogeny broth (LB) at 37° C. until OD600 reaches 0.5-0.8. The culture was then induced with 0.5 mM isopropyl β-d-1-thiogalactopyranoside (IPTG, Millipore Sigma, Cat#: I6758-10G) at 18° C. for 20 hours. Following induction, the cells were centrifuged, lysed, sonicated, and further centrifuged to obtain the cell lysate that contains the desired his-tagged protein. The lysate was then incubated with cobalt resin (Thermo Fisher Scientific, Cat#: 89964) followed by elution with 150 mM imidazole (Millipore Sigma, Cat#: I5513) and eventually buffer exchanged to 20 mM Hepes, 150 mM NaCl, 10% glycerol with 1 mM dithiothreitol (DTT, Millipore Sigma Cat#: 10197777001), aliquoted and stored in −80 ° C. freezer for future use.

In vitro STING Signaling Activation in 293T Reporter Cells

The STING deficient human embryonic kidney (HEK293T) cell line was used to study the capability of restoring STING signaling in vitro. The assay was based on our previous published protocol⁸⁶. Briefly, a reporter cell line was generated from transfecting HEK293T cells with pGL4.45 [luc2p/ISRE/Hygro] plasmid (Promega), which was able to express luciferase driven by the interferon-sensitive response element (ISRE) stimulated by STING signaling. 100 μL of 3×10⁵ cells/mL HEK293T-luc2p/ISRE/Hygro cells were seeded in 96 well plate in Dulbecco's modified Eagle's medium (DMEM, Corning, Cat#: 10-041-CV) supplemented with 10% fetal bovine serum (FBS, Gibco, Cat#: 10437-028) and 1% penicillin/streptomycin (Corning, Cat#: 30-002-CI). After an overnight incubation, 0.025 μg cGAMP (Invivogen, Cat#: tlrl-nacga23-02) mixed with 1.7 μg ICB-ΔTM protein and 1.7 μL TransIT-X2 commercial transfection reagent (Mirus, Cat#: MIR6004) were mixed in 20 μL Opti-MEM™ medium (Gibco, Cat#: 31985062), incubated for 15 minutes for protein complex formation before added to the wells. 24 hours post treatment, cells were lysed for firefly luciferase assay (Biotium, Cat#: 30075-2) to quantify the interferon-luciferase activity.

Ex vivo Cytotoxicity Assay with MC38-SIINFEKL Cells

MC38-SIINFEKL cells were created via lenti-viral vector transduction. pLenti-CMV-GFP-Puro™ plasmid was obtained from Addgene (Cat#: 17448). The DNA sequence encoding for SIINFEKL peptide was inserted at the N-terminus of GFP via mutagenesis. HEK293T cells were transfected with pLenti-CMV-SIINFEKL-GFP-Puro plasmid with the help of TransIT-X2 transfection reagent. The culture medium containing lentivirus were collected, filtered through 0.45 μm filter and added to plated MC38 cells with 5 μg/mL polybrene (Millipore Sigma, Cat#: H9268), followed by puromycin (Millipore Sigma, Cat#: P9620) selection in the concentration range of 5-20 μg/mL. The final transduction efficiency was confirmed with flow cytometry based on percentage GFP expression.

To perform the ex vivo cytotoxicity assay of CD8⁺ T cells against cancer cells, MC38-SIINFEKL cells were first treated with 50 ng/mL mouse IFNγ for 24 hours to induce PDL1 upregulation, which is confirmed by qPCR to be approximately 10-fold of untreated cells' PDL1 expression level. In parallel, splenocytes were harvested from C57BL/6-Tg(TcraTcrb)1100Mjb/J (OT1) mice and activated with αCD3/CD28 Dynabeads™ (Gibco, Cat#: 11456D) at a density of 2×10⁶ cells/mL Roswell Park Memorial Institute medium (RPMI, Corning, Cat#: 10-013-CV) according to the instruction manual. The IFNγ treated MC38-SIINFEKL cells were then washed and plated at a density of 4×10⁵ cells/mL with stimulated OT1 splenocytes at a ratio of 1:2 in 1 mL RPMI medium in 12 well plates for 48 hours, with the addition of 17 μg ΔPDL1-ΔTM plus 0.25 μg cGAMP, 17 μg α96G3M-ΔTM plus 0.25 μg cGAMP, 16 μg ΔPDL1 mono antibody (clone 10F.9G2, BioXCell, Cat#: BE0101), along with an untreated control. After 48 hours of coculture, cells were stained with Zombie Aqua™ live-dead stain (Biolegend, Cat#: 423101) and analyzed with flow cytometry. MC38 tumor cells were gated with FITC channel due to their GFP expression, and their percentage viability were used to assess the ex vivo PDL1 checkpoint blockade efficiency.

### Tumor Inoculation and Intratumoral Treatment

MC38 and YUMM1.7 tumors were inoculated in C57BL/6 or B6(Cg)-Sting1tm1.2Camb/J (STING knockout) mice by subcutaneously (s.c.) injecting 10⁶ cells suspended in 100 μL sterile phosphate buffered saline (PBS, Lonza, Cat#: 17-516F) into the shaved hind right flank. All C57BL/6 mice were female and purchased at the age of 8 weeks old, tumors were usually inoculated 1-2 weeks after mice arrival at the lab. STING knock-out mice were bred in house, for tumor treatment studies, each group contained the equal number of male and female mice of the same age (8-12 weeks old). For rechallenge, 10⁵ cells in 100 μL sterile PBS were injected into the hind left flank. The tumor sizes were monitored every two days with caliper measurement, volumes calculated as length×width² then divided by 2. Treatment by intratumoral (i.t.) injections started when the average tumor volume reached around 100 mm³. Each i.t. injection consisted of 170 μg of ICB-ΔTM protein with or without 2.5 μg cGAMP. For ICB antibody controls, each i.t. injection consists of 80 μg ΔPDL1 mAb (clone 10F.9G2, BioXCell, Cat#: BE0101) and 80 μg αCTLA4 mAb (clone 9D9, BioXCell, Cat#: BE0164). The humane endpoint tumor burden for euthanasia of all three mouse models was 1000 mm³.

4T1 cells were inoculated in Balb/c mice in the mammary fat pad. A small incision was made next to the 4^{th} nipple, 10⁶ 4T1 cells suspended in 50 μL PBS were injected into the mammary fat pad through the incision, which is subsequently closed with suture (Ethicon, Cat#: R690G). Caliper measurement and intratumoral injections were performed similar to MC38 and YUMM1.7 models. The only caution was that intratumoral injections for 4T1 tumors were done very slowly to achieve a uniform distribution of drug inside the tumor. Fast injections would result in a pouch of liquid seeping outside the tumor.

### Immune Cell Depletion in Mice

Depletion of mouse immune cell populations were carried out according to the literature^{75,87} by intraperitoneally (i.p.) injecting mice with antibodies against mouse CD8α (clone 2.43, BioXCell, Cat#: BP0061, 400 μg twice every week), CD4 (clone GK1.5, BioXCell, Cat#: BP0003-1, 400 μg twice every week), NK1.1 (clone PK136, BioXCell, Cat#: BE0036, 400 μg twice every week), or CSF1R (clone AFS98, BioXCell, Cat#: BP0213, 300 μg every other day). 36 hours post i.p. injection, mice PBMCs were sampled via cheek bleeding and stained for flow cytometry analysis to confirm the depletion efficiency.

Tumor Digestion for Immune Cell Analysis with Flow Cytometry

Approximately 100 mg mouse tumor was resected, weighed, and then minced to small pieces with diameters <1 mm in 500 μL PBS with 1 mg/mL collagenase type IV (Gibco, Cat#: 17104019). The minced tumor in collagenase was then incubated at 37° C. under shaking for 20 mins (MC38 tumors) or 30 mins (YUMM1.7 and 4T1 tumors). After incubation, the mixture was diluted with 1 mL PBS, vortexed, and filtered through 70 μm nylon mesh cell strainer (Fisherbrand, Cat#: 22363548). In case there were a lot of red blood cells (RBC), the filtrate was resuspended in 5 mL of RBC lysis buffer (Millipore Sigma, Cat#: R7767-100 mL), incubated at room temperature for 5 mins, then pelleted with centrifugation. The filtrate was then washed twice with PBS, resuspended in 10 mL PBS, and sampled for cell count. For intracellular cytokine staining samples, an additional 4 hour 37° C. incubation was performed prior to staining in RPMI supplemented with non-essential amino acid (NEAA, Gibco, Cat#: 11140050) and Golgi inhibitor (GolgiStop™, BD Biosciences, Cat#: 554724).

Afterwards, the cells were collected and washed with PBS for FACS staining. 10⁶ cells from each tumor samples were loaded into 96 V-bottom well plate in 50 μL PBS and first stained with Zombie Aqua™ live/dead dye, followed by 20 min incubation on ice avoiding light. The cells are then washed with FACS buffer (PBS with 1% BSA and 2 mM EDTA). After washing, each well of cells are stained with Fc-blocker anti-mouse CD16/32 (Thermo Fisher Scientific, Cat#: 14-9161-73) in 50 μL FACS buffer, followed by 15 min incubation on ice. When the blocking was complete, surface staining antibodies were added to the cells for 30 min incubation on ice, followed by two washes with FACS buffer. Intracellular cytokine staining was then performed with the BD Cytofix/Cytoperm™ kit (BD Biosciences, Cat #555028). 100 μL fixation/permeabilization solution was first added to each well followed by 20 min incubation on ice. The cells were then washed twice with Perm/wash buffer and stained with intracellular staining antibodies in 50 μL Perm/wash buffer for 30 min on ice. After staining, the cells were washed twice with Perm/wash buffer, resuspended in FACS buffer, and analyzed with LSR-II-Fortessa flow cytometer (BD Biosciences). Data were analyzed with FlowJo™ software.

Flow cytometry antibodies (all from Biolegend) used are anti-mouse CD4 (clone GK1.5, Cat#: 100434), CD3 (clone 17A2, Cat#: 100204 and 100232), CD8α (clone 53-6.7, Cat#: 100707), F4/80 (clone BM8, Cat#: 123116 and 123135), NK1.1 (clone S17016D, Cat#: 156514), NKp46 (clone 29A1.4, Cat#: 137618), Ly6C (clone HK1.4, Cat#: 128031), Ly6G (clone 1A8, Cat#: 127645), CD45 (clone 30-F11, Cat#: 103128), CD11b (clone M1/70, Cat#: 101241), IFNγ (clone XMG1.2, Cat#: 505813), IL4 (clone 11B11, Cat#: 504133), PU.1 (clone 7C2C34, Cat#: 681307), IL17 (clone TC11-18H10.1, Cat#: 506915), FoxP3 (clone MF-14, Cat#: 126419), granzyme B (clone GB11, Cat#: 515403), and Perforin (clone S16009B, Cat#: 154404).

### Tumor Protein Extraction for Cytokine Analysis

Approximately 50 mg mouse tumor was resected and grinded with microcentrifuge tube pestles in T-PER buffer (Thermo Fisher Scientific, Cat#: 78510) with protease inhibitor (Thermo Fisher Scientific, Cat#: 78425). The protein extraction solutions were then centrifuged at 14,000×g for 15 min. Supernatant protein concentrations were quantified with Nanodrop based on absorption at 280 nm and were all diluted to 5 mg/mL. Samples were then frozen and shipped to Eve Technology for multiplex assay for the cytokine concentrations. Heat maps of the protein levels were plotted as log₂(fold change of untreated groups), with hierarchical clustering based on one minus Pearson correlation with complete linkage method performed with Morpheus software.

### Splenocyte CD4⁺ T Cell Isolation and Phenotyping

Spleen from C57BL/6 mice were first grinded and filtered through 70 μm cell strainer and washed once by RBC lysis buffer. The cells were then washed and resuspended in FACS buffer. EasySep™ Mouse CD4⁺ T cell isolation kit (Stemcell Technology, Cat#: 19852) was used to isolate CD4⁺ T cells from splenocytes. Splenocytes were re-suspended in PBS containing 2% FBS and 1 mM EDTA at the concentration of 10⁸ cells/mL. Per mL of the splenocytes, 50 μL rat serum and 50 μL isolation cocktail were added followed by a 10 mins incubation at room temperature. 75 μL of vortexed magnetic beads Rapidspheres™ was added per mL of sample, mixed and incubated for 2.5 mins. Then buffer was added to the sample to top it up to 2.5 mL, before placed in the magnet (EasySep™) for 2.5 mins. Afterwards, the separated CD4⁺ T cells were poured out in one continuous motion. The separation effect is verified with flow cytometry with anti-CD4 surface staining. For phenotyping, the isolated CD4⁺ T cells were then treated with ICB-ΔTM protein mixed with cGAMP in RPMI media with NEAA for 8 hours (GolgiStop™ added for the last 4 hours) before staining for flow cytometry analysis as described in the tumor cell FACS section.

### Immunocytochemistry

CD4⁺ T cells treated with Cy5-labeled ICB-ΔTM protein mixed with cGAMP were collected in microcentrifugation tubes along with untreated controls. The cells were washed 3 times with PBS, fixed with PBS containing 4% formaldehyde (Millipore Sigma, Cat#: 47608-250ML-F) for 15 mins on ice, then permeabilized by PBS containing 0.1% Triton X-100 (Millipore Sigma, Cat#: T8787) on ice for 10 mins. Afterwards, cells were washed by PBS with 0.05% Tween20 (Millipore Sigma, Cat#: P9416) and 1% BSA and stained with Alexa™ Flour 488 phalloidin (Invitrogen, Cat#: A12379) and Hoechst dye (Thermo Fisher Scientific, Cat#: 62249) in the same buffer for 30 mins avoiding light. The cells were then washed 3 times with PBS containing 0.05% Tween20 and 1% BSA and loaded onto microscope slides with anti-fade mounting media and covered with cover slips. Images were acquired with Olympus FV1200 confocal microscope and analyzed with ImageJ software.

### Organ Biodistribution

C57BL/6 mice inoculated with YUMM1.7 tumor were injected i.t. with 170 μg Cy5-labeled anti-GFP/PDL1/CTLA4-ΔTM protein mixed with 2.5 μg cGAMP in 25 μL PBS. 24 hours post treatment, mice were sacrificed and organs plus tumors and both inguinal lymph nodes were collected for fluorescent imaging with the In Vivo Imaging System (IVIS, Xenogen). Data analysis was performed with the Living Image software (Xenogen).

### IFNγ ELISPOT

PBMCs were obtained from cheek bleeding of the immune cell depleted mice collected with EDTA-coated collection tubes (Greiner Bio-one™, K3EDTA Cat#: 450530). Whole blood was washed twice with RBC lysis buffer to obtain peripheral blood mononuclear cells (PBMCs). ELISPOT was performed according to the kit instruction manual (BD ELISPOT reagent kit). Briefly, ELISPOT plate was first coated with anti-IFNγ capture antibodies overnight. The following day, the plate was washed and blocked. Equal number of PBMCs from each mouse were co-cultured with X-ray irradiated YUMM1.7 cells in the plate overnight. On the third day, cell suspension was aspirated, wells were incubated with detection antibody, streptavidin-HRP and substrate solution with 5 washes between every incubation. Spots were enumerated with an ELISPOT plate reader.

### Statistical Analysis

Data were analyzed with Prism GraphPad™: tumor volume and body weight change were analyzed with two-way ANOVA, the survival curve was analyzed with Kaplan-Meier, the remaining plots were analyzed with one-way ANOVA. Outlier were analyzed and removed with the Grubb's test.

### References

1. Carlino, M. S., Larkin, J. & Long, G. V. Immune checkpoint inhibitors in melanoma. The Lancet 398, 1002-1014 (2021).
2. Sharma, P. & Allison, J. P. The future of immune checkpoint therapy. Science 348, 56-61 (2015).
3. Zou, W., Wolchok, J. D. & Chen, L. PD-L1 (B7-H1) and PD-1 pathway blockade for cancer therapy: Mechanisms, response biomarkers, and combinations. Science translational medicine 8, 328rv324-328rv324 (2016).
4. Emambux, S., Tachon, G., Junca, A. & Tougeron, D. Results and challenges of immune checkpoint inhibitors in colorectal cancer. Expert Opinion on Biological Therapy 18, 561-573 (2018).
5. Golshani, G. & Zhang, Y. Advances in immunotherapy for colorectal cancer: a review. Therapeutic advances in gastroenterology 13, 1756284820917527 (2020).
6. Zhou, Y. et al. First-line treatment for patients with advanced non-small cell lung carcinoma and high PD-L1 expression: pembrolizumab or pembrolizumab plus chemotherapy. Journal for immunotherapy of cancer 7, 120 (2019).
7. Shields, M. D., Marin-Acevedo, J. A. & Pellini, B. Immunotherapy for Advanced Non-Small Cell Lung Cancer: A Decade of Progress. American Society of Clinical Oncology Educational Book, e105-e127 (2021).
8. Jenkins, R. W., Barbie, D. A. & Flaherty, K. T. Mechanisms of resistance to immune checkpoint inhibitors. British journal of cancer 118, 9-16 (2018).
9. Haslam, A. & Prasad, V. Estimation of the Percentage of US Patients With Cancer Who Are Eligible for and Respond to Checkpoint Inhibitor Immunotherapy Drugs. JAMA network open 2, e192535 (2019).
10. Patel, S. A. & Minn, A. J. Combination Cancer Therapy with Immune Checkpoint Blockade: Mechanisms and Strategies. Immunity 48, 417-433 (2018).
11. Nishino, M., Ramaiya, N. H., Hatabu, H. & Hodi, F. S. Monitoring immune-checkpoint blockade: response evaluation and biomarker development. Nature reviews Clinical oncology 14, 655-668 (2017).
12. Bagchi, S., Yuan, R. & Engleman, E. G. Immune checkpoint inhibitors for the treatment of cancer: clinical impact and mechanisms of response and resistance. Annual Review of Pathology: Mechanisms of Disease 16, 223-249 (2021).
13. Lu, L. et al. Clinically approved combination immunotherapy: Current status, limitations, and future perspective. Current Research in Immunology 3, 118-127 (2022).
14. Barbari, C. et al. Immunotherapies and Combination Strategies for Immuno-Oncology. International journal of molecular sciences 21 (2020).
15. Varayathu, H., Sarathy, V., Thomas, B. E., Mufti, S. S. & Naik, R. Combination Strategies to Augment Immune Check Point Inhibitors Efficacy—Implications for Translational Research. Frontiers in oncology 11, 559161 (2021).
16. Wolchok, J. D. et al. Overall Survival with Combined Nivolumab and Ipilimumab in Advanced Melanoma. New England Journal ofMedicine 377, 1345-1356 (2017).
17. Larkin, J. et al. Combined Nivolumab and Ipilimumab or Monotherapy in Untreated Melanoma. New England Journal of Medicine 373, 23-34 (2015).
18. Vafaei, S. et al. Combination therapy with immune checkpoint inhibitors (ICIs); a new frontier. Cancer Cell International 22, 2 (2022).
19. Grosser, R., Cherkassky, L., Chintala, N. & Adusumilli, P. S. Combination Immunotherapy with CAR T Cells and Checkpoint Blockade for the Treatment of Solid Tumors. Cancer cell 36, 471-482 (2019).
20. Huemer, F. et al. Combination Strategies for Immune-Checkpoint Blockade and Response Prediction by Artificial Intelligence. International journal of molecular sciences 21 (2020).
21. Dongye, Z., Li, J. & Wu, Y. Toll-like receptor 9 agonists and combination therapies:

strategies to modulate the tumour immune microenvironment for systemic anti-tumour immunity. British Journal of Cancer (2022).
22. Sokolowska, O. et al. Immune checkpoint inhibition improves antimyeloma activity of bortezomib and STING agonist combination in Vk*MYC preclinical model. Clinical and experimental medicine (2022).
23. Ghaffari, A. et al. STING agonist therapy in combination with PD-1 immune checkpoint blockade enhances response to carboplatin chemotherapy in high-grade serous ovarian cancer. Br J Cancer 119, 440-449 (2018).
24. Chon, H. Effect of STING agonist on tumor immune microenvironment of non-inflamed lung cancer and efficacy of immune checkpoint blockade. Journal of Clinical Oncology 36, 178-178 (2018).
25. Garland, K. M., Sheehy, T. L. & Wilson, J. T. Chemical and Biomolecular Strategies for STING Pathway Activation in Cancer Immunotherapy. Chemical Reviews (2022).
26. Motedayen Aval, L., Pease, J. E., Sharma, R. & Pinato, D. J. Challenges and Opportunities in the Clinical Development of STING Agonists for Cancer Immunotherapy. Journal of clinical medicine 9 (2020).
27. Li, K. et al. The lipid platform increases the activity of STING agonists to synergize checkpoint blockade therapy against melanoma. Biomaterials science 9, 765-773 (2021).
28. Cheng, N. et al. A nanoparticle-incorporated STING activator enhances antitumor immunity in PD-L1—insensitive models of triple-negative breast cancer. JCI insight 3 (2018).
29. Wu, J. & Chen, Z. J. Innate immune sensing and signaling of cytosolic nucleic acids. Annu Rev Immunol 32, 461-488 (2014).
30. Borden, E. C. Interferons α and β in cancer: therapeutic opportunities from new insights. Nature Reviews Drug Discovery 18, 219-234 (2019).
31. Marcus, A. et al. Tumor-derived cGAMP triggers a STING-mediated interferon response in non-tumor cells to activate the NK cell response. Immunity 49, 754-763. e754 (2018).
32. Nicolai, C. J. et al. NK cells mediate clearance of CD8+ T cell-resistant tumors in response to STING agonists. Science immunology 5, eaaz2738 (2020).
33. Berger, G. et al. STING activation promotes robust immune response and NK cell-mediated tumor regression in glioblastoma models. Proceedings of the National Academy of Sciences 119, e2111003119 (2022).
34. Shakya, A K., Lee, C. H., Uddin, M. J. & Gill, H. S. Assessment of Th1/Th2 Bias of STING Agonists Coated on Microneedles for Possible Use in Skin Allergen Immunotherapy. Molecular pharmaceutics 15, 5437-5443 (2018).
35. Rossi, M. et al. STING Agonist Combined to a Protein-Based Cancer Vaccine Potentiates Peripheral and Intra-Tumoral T Cell Immunity. Frontiers in immunology 12 (2021).
36. Van Herck, S., Feng, B. & Tang, L. Delivery of STING agonists for adjuvanting subunit vaccines. Advanced Drug Delivery Reviews 179, 114020 (2021).
37. Benoit-Lizon, I. et al. CD4 T cell-intrinsic STING signaling controls the differentiation and effector functions of TH1 and TH9 cells. Journal for immunotherapy of cancer 10 (2022).
38. Kim, H.-J. & Cantor, H. CD4 T-cell Subsets and Tumor Immunity: The Helpful and the Not-so-Helpful. Cancer Immunology Research 2, 91-98 (2014).
39. Lee, J. et al. The Multifaceted Role of Th1, Th9, and Th17 Cells in Immune Checkpoint Inhibition Therapy. Frontiers in immunology 12 (2021).
40. Immunovative Therapies, L. Immunotherapy for Third Line Metastatic Colorectal Cancer (STIMVAX). 2022 Aug. 31, 2022 clinicaltrials.gov/ct2/show/NCT04444622
41. Laidlaw, B. J., Craft, J. E. & Kaech, S. M. The multifaceted role of CD4+ T cells in CD8+ T cell memory. Nature Reviews Immunology 16, 102-111 (2016).
42. Tay, R. E., Richardson, E. K. & Toh, H. C. Revisiting the role of CD4+ T cells in cancer immunotherapy—new insights into old paradigms. Cancer Gene Therapy 28, 5-17 (2021).
43. Mersana Therapeutics, I. Mersana Therapeutics Announces FDA Grant of Orphan Drug Designation to XMT-2056 for the Treatment of Gastric Cancer. 2022.
44. Meric-Bemstam, F. et al. Phase Ib study of MIW815 (ADU-S100) in combination with spartalizumab (PDR001) in patients (pts) with advanced/metastatic solid tumors or lymphomas. Journal of Clinical Oncology 37, 2507-2507 (2019).
45. Rossi, M. et al. STING Agonist Combined to a Protein-Based Cancer Vaccine Potentiates Peripheral and Intra-Tumoral T Cell Immunity. Frontiers in immunology 12, 695056 (2021).
46. Melero, I., Castanon, E., Alvarez, M., Champiat, S. & Marabelle, A. Intratumoural administration and tumour tissue targeting of cancer immunotherapies. Nature Reviews Clinical Oncology 18, 558-576 (2021).
47. Marabelle, A., Tselikas, L., de Baere, T. & Houot, R. Intratumoral immunotherapy: using the tumor as the remedy. Annals of Oncology 28, xii33-xii43 (2017).
48. Patel, S. et al. The Common R71H-G230A-R293Q Human TMEM173 Is a Null Allele. Journal of immunology (Baltimore, Md.: 1950) 198, 776-787 (2017).
49. Ruiz-Moreno, J. S. et al. The common HAQ STING variant impairs cGAS-dependent antibacterial responses and is associated with susceptibility to Legionnaires' disease in humans. PLoS pathogens 14, e1006829 (2018).
50. Kennedy, R. B. et al. Polymorphisms in STING Affect Human Innate Immune Responses to Poxviruses. Frontiers in immunology 11, 567348 (2020).
51. Walker, M. M. et al. Selective Loss of Responsiveness to Exogenous but Not Endogenous Cyclic-Dinucleotides in Mice Expressing STING-R231H. Frontiers in immunology 11, 238 (2020).
52. He, Y. et al. Self-assembled cGAMP-STINGΔTM signaling complex as a bioinspired platform for cGAMP delivery. Science advances 6, eaba7589 (2020).
53. He, Y. et al. Peptide-based Cancer Vaccine Delivery via the STINGΔTM-cGAMP Complex. Advanced Healthcare Materials, 2200905.
54. Lin, Y., Xu, J. & Lan, H. Tumor-associated macrophages in tumor metastasis: biological roles and clinical therapeutic applications. Journal of Hematology & Oncology 12, 76 (2019).
55. Kiniwa, T. et al. NK cells activated by Interleukin-4 in cooperation with Interleukin-15 exhibit distinctive characteristics. Proceedings of the National Academy of Sciences 113, 10139-10144 (2016).
56. Tie, Y., Tang, F., Wei, Y.-q. & Wei, X.-w. Immunosuppressive cells in cancer: mechanisms and potential therapeutic targets. Journal of Hematology & Oncology 15, 61 (2022).
57. LLC, M. S. D. Study of Ulevostinag (MK-1454) Alone or in Combination With Pembrolizumab (MK-3475) in Participants With Advanced/Metastatic Solid Tumors or Lymphomas (MK-1454-001). 2022 Jun. 13, 2022 clinicaltrials.gov/ct2/show/results/NCT03010176
58. Pharmaceuticals, N. Study of the Safety and Efficacy of MIW815 With PDR001 in Patients With Advanced/Metastatic Solid Tumors or Lymphomas. 2022 May 3, 2022 clinicaltrials.gov/ct2/show/NCT03172936
59. Amouzegar, A., Chelvanambi, M., Filderman, J. N., Storkus, W. J. & Luke, J. J. STING Agonists as Cancer Therapeutics. Cancers 13 (2021).
60. Rosenberg, J. & Huang, J. CD8(+) T Cells and NK Cells: Parallel and Complementary Soldiers of Immunotherapy. Current opinion in chemical engineering 19, 9-20 (2018).
61. Alspach, E. et al. MHC-II neoantigens shape tumour immunity and response to immunotherapy. Nature 574, 696-701 (2019).
62. Wculek, S. K. et al. Dendritic cells in cancer immunology and immunotherapy. Nature reviews. Immunology 20, 7-24 (2020).
63. Sato, Y. et al. CD4+ T cells induce rejection of urothelial tumors after immune checkpoint blockade. JCI insight 3 (2018).
64. Alspach, E. et al. MHC-II neoantigens shape tumour immunity and response to immunotherapy. Nature 574, 696-701 (2019).
65. Shakya, A. K., Lee, C. H., Uddin, M. J. & Gill, H. S. Assessment of Th1/Th2 Bias of sting agonists coated on microneedles for possible use in skin allergen immunotherapy. Molecular pharmaceutics 15, 5437-5443 (2018).
66. Kocabas, B. B. et al. Dual-adjuvant effect of pH-sensitive liposomes loaded with STING and TLR9 agonists regress tumor development by enhancing Th1 immune response. Journal of Controlled Release 328, 587-595 (2020).
67. Ulrich-Lewis, J. T. et al. STING is required in conventional dendritic cells for DNA vaccine induction of type IT helper cell-dependent antibody responses. Frontiers in immunology 13 (2022).
68. Chen, Daniel S. & Mellman, I. Oncology Meets Immunology: The Cancer-Immunity Cycle. Immunity 39, 1-10 (2013).
69. Bald, T., Krummel, M. F., Smyth, M. J. & Barry, K. C. The NK cell-cancer cycle: advances and new challenges in NK cell-based immunotherapies. Nature immunology 21, 835-847 (2020).
70. Bihl, F., Germain, C., Luci, C. & Braud, V. M. Mechanisms of NK cell activation: CD4+ T cells enter the scene. Cellular and Molecular Life Sciences 68, 3457 (2011).
71. Kumamoto, Y., Mattei, L. M., Sellers, S., Payne, G. W. & Iwasaki, A. CD4+ T cells support cytotoxic T lymphocyte priming by controlling lymph node input. Proceedings of the National Academy of Sciences of the United States of America 108, 8749-8754 (2011).
72. Borst, J., Ahrends, T., Ba̧bala, N., Melief, C. J. & Kastenmüller, W. CD4+ T cell help in cancer immunology and immunotherapy. Nature Reviews Immunology 18, 635-647 (2018).
73. Jing, W., Gershan, J. A. & Johnson, B. D. Depletion of CD4 T cells enhances immunotherapy for neuroblastoma after syngeneic HSCT but compromises development of antitumor immune memory. Blood, The Journal of the American Society of Hematology 113, 4449-4457 (2009).
74. Kim, S.-H., Han, C., Kwon, B. S. & Choi, B. K. CD4 depletion potentiates anti-tumor immunity in adoptive immunotherapy by increasing IL-18Rαhi endogenous CD8+ T cells. Am Assoc Immnol; 2020.
75. Li, Y. et al. Multifunctional oncolytic nanoparticles deliver self-replicating IL-12 RNA to eliminate established tumors and prime systemic immunity. Nature Cancer 1, 882-893 (2020).
76. Nagai, H. et al. Elimination of CD4+ T cells enhances anti-tumor effect of locally secreted interleukin-12 on B16 mouse melanoma and induces vitiligo-like coat color alteration. Journal of investigative dermatology 115, 1059-1064 (2000).
77. Ueha, S. et al. Robust Antitumor Effects of Combined Anti-CD4-Depleting Antibody and Anti-PD-1/PD-L1 Immune Checkpoint Antibody Treatment in MiceAnti-CD4 and Immune Checkpoint Antibody Synergy. Cancer immunology research 3, 631-640 (2015).
78. Shitara, K. et al. First-in-human phase 1 study of IT1208, a defucosylated humanized anti-CD4 depleting antibody, in patients with advanced solid tumors. Journal for immunotherapy of cancer 7, 1-11 (2019).
79. Antonelli, A. C., Binyamin, A., Hohl, T. M., Glickman, M. S. & Redelman-Sidi, G. Bacterial immunotherapy for cancer induces CD4-dependent tumor-specific immunity through tumor-intrinsic interferon-γ signaling. Proceedings of the National Academy of Sciences 117, 18627-18637 (2020).
80. Kuhl, N. et al. STING agonism turns human T cells into interferon-producing cells but impedes their functionality. EMBO reports, e55536 (2023).
81. Cerboni, S. et al. Intrinsic antiproliferative activity of the innate sensor STING in T lymphocytes. Journal of Experimental Medicine 214, 1769-1785 (2017).
82. Luan, Y.-y., Zhang, L., Peng, Y.-q., Li, Y.-y. & Yin, C.-h. STING modulates necrotic cell death in CD4 T cells via activation of PARP-1/PAR following acute systemic inflammation. International Immunopharmacology 109, 108809 (2022).
83. Li, Y. et al. Prebiotic-induced anti-tumor immunity attenuates tumor growth. Cell reports 30, 1753-1766. e1756 (2020).
84. Agarwal, Y. et al. Intratumourally injected alum-tethered cytokines elicit potent and safer local and systemic anticancer immunity. Nature Biomedical Engineering 6, 129-143 (2022).
85. Jailkhani, N. et al. Noninvasive imaging of tumor progression, metastasis, and fibrosis using a nanobody targeting the extracellular matrix. Proceedings of the National Academy of Sciences 116, 14181-14190 (2019).
86. He, Y., Hong, C., Irvine, D. J., Li, J. & Hammond, P. T. In vitro STING Activation with the cGAMP-STINGΔTM Signaling Complex. Bio-protocol 11, e3905-e3905 (2021).
87. Moynihan, K. D. et al. Eradication of large established tumors in mice by combination immunotherapy that engages innate and adaptive immune responses. Nature medicine 22, 1402-1410 (2016).

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A fusion protein, comprising a STINGΔTM protein fused to a nanobody.

2. The fusion protein of claim 1, wherein the STINGΔTM comprises an amino acid sequence with at least 75%, 80%, 85%, 90%, 95%, 96%, 97%, 98%, 99% or 100% identity to the amino acid sequence selected from SEQ ID NO: 3-6.

3. The fusion protein of claim 1, wherein the nanobody is fused to the N-terminus of the STINGΔTM.

4. The fusion protein of claim 1, wherein the nanobody is capable of binding to a cancer cell or tumor extracellular matrices.

5. The fusion protein of claim 4, wherein the nanobody is capable of binding to Cytotoxic T-Lymphocyte Associated Protein 4 (CTLA4), Programmed death protein 1 (PD-1), Programmed death-ligand 1 (PD-L1), Programmed death-ligand 2 (PD-L2), adenosine A2a receptor (A2AR), B7 homolog 3 protein (B7-H3), B7 homolog 4 protein (B7-H4), B- and T-lymphocyte attenuator (BTLA), killer cell immunoglobulin-like receptor (KIR), Lymphocyte-activation gene 3 (LAG3), T cell immunoglobulin and mucin domain-containing protein 3 (TIM-3), Fibronectin extra type III B module (EIIIB), T cell immunoreceptor with Ig and ITIM domains (TIGIT), Natural killer group 2, member A (NGK2A), P-selectin glycoprotein ligand-1 (PSGL-1), or V-domain immunoglobulin suppressor of T-cell activation (VISTA).

6. The fusion protein of claim 4, wherein the nanobody is capable of binding to CTLA4 or PD-L1.

7. The fusion protein of claim 6, wherein the nanobody comprises an amino acid sequence with at least 75%, 80%, 85%, 90%, 95%, 96%, 97%, 98%, 99% or 100% identity to the amino acid sequence selected from the group consisting of SEQ ID NO: 7-10.

8. The fusion protein of claim 1, comprising an amino acid sequence with at least 75%, 80%, 85%, 90%, 95%, 96%, 97%, 98%, 99% or 100% identity to the amino acid sequence selected from SEQ ID NO: 11-42, wherein residues in parentheses are optional and may be present or may be deleted in whole or in part.

9. A nucleic acid encoding the fusion protein of claim 1.

10. An expression vector comprising the nucleic acid of claim 9 operatively linked to a suitable control sequence.

11. A host cell comprising the expression vector of claim 10.

12. A composition, comprising the fusion protein of claim 1 and a STING agonist.

13. The composition of claim 12, wherein the STING agonist is a cytosolic cyclic dinucleotide (CDN).

14. The composition of claim 13, wherein the CDN is c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP.

15. The composition of claim 12, wherein the STING agonist is a non-nucleotidyl small molecule.

16. The composition of claim 15, wherein the non-nucleotidyl small molecule is 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo[3,2-a:3′,2′-d]pyrazine-5,10(5aH,10aH)-dione, 4-(2-chloro-6-fluorobenzyl)-N-(furan-2-yl methyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3-b][1,4]thiazine-7-carboxamide, 2-oxo-1,2,3,4-tetrahydroquinoline-7-carboxamide, or 2-Oxo-1,2,3,4-tetrahydroquinazoline-7-carboxamides.

17. A method of treating cancer or an infectious disease, comprising administering to a patient in need thereof an effective amount of the composition of claim 12.

18. The method of claim 17, wherein the method is for treating cancer.

19. The method of claim 18, wherein the administering comprises intratumoral injection.

20. The method of claim 18, wherein the cancer comprises a colon carcinoma, a melanoma, or breast cancer.

21. The fusion protein of claim 1, further comprising a cell penetrating peptide.
